(19) United States
(12) Patent Application Publication
(10) Pub. No.: US 2006/0020155 A1
(43) Pub. Date: Jan. 26, 2006

(54) Processes for converting oxygenates to olefins at reduced volumetric flow rates

(76) Inventors: James H. Beech JR., Kingwood, TX (US); Michael P. Nicoletti, Houston, TX (US); Cor F. Van Egmond, Pasadena, TX (US)

Correspondence Address: ExxonMobil Chemical Company;Law Technology, P.O. Box 2149, Baytown, TX, 77522-2149, US

(21) Appl. No.: 10/897,198

(22) Filed: Jul. 21, 2004

(51) Int. Cl. C07C 1/00

(52) U.S. Cl. 585/639

(57) ABSTRACT

This invention provides processes for forming light olefins from methanol and/or from syngas through a dimethyl ether intermediate. Specifically, the invention is to converting methanol and/or syngas to dimethyl ether and water in the presence of a first catalyst, preferably comprising γ-alumina, and converting the dimethyl ether to light olefins and water in the presence of a second catalyst, preferably a molecular sieve catalyst composition.

### FIELD OF THE INVENTION

The present invention relates to processes for forming light olefins. More particularly, the invention relates to converting methanol or syngas to dimethyl ether, which is then converted to the light olefins.

### BACKGROUND OF THE INVENTION

Light olefins, defined herein as ethylene and propylene, separately or in combination, are important commodity petrochemicals useful in a variety of processes for making plastics and other chemical compounds. Ethylene is used to make various polyethylene plastics, and in making other chemicals vinyl chloride, ethylene oxide, ethyl benzene and alcohol. Propylene is used to make various polypropylene plastics, and in making other chemicals such as acrylonitrile and propylene oxide.

The petrochemical industry has known for some time that oxygenates, especially alcohols, are convertible into light olefins. The preferred conversion process is generally referred to as an oxygenate to olefin (OTO) reaction process. Specifically, in an OTO reaction process, an oxygenate contacts a molecular sieve catalyst composition under conditions effective to convert at least a portion of the oxygenate to light olefins. When methanol is the oxygenate, the process is generally referred to as a methanol to olefin (MTO) reaction process. Methanol is a particularly preferred oxygenate for the synthesis of ethylene and/or propylene.

In order to be commercially viable, a commercial OTO reaction system utilizing methanol as the primary oxygenate feed must produce a very large volumetric flow of reactor effluent at olefin production capacities. As a result, a MTO reactor may require a very large disengaging vessel to separate catalyst from the reactor effluent. Ethylene production capacities of about 1,000 KTA from a methanol feedstock, for example, may require a single reactor having a disengaging vessel diameter of over 60 feet. Such vessel diameters are well in excess of what can be shop fabricated and therefore must be fabricated in the field, resulting in significant expense. Additionally, the high volume of effluent also entrains larger quantities of expensive molecular sieve catalyst, which are lost from the process and result in a further increase in operating cost. The high effluent volumes are caused by the formation of unwanted water by-products in the effluent, which can comprise as much as about 70 mole percent of the entire effluent depending on the feed water content. These high water concentrations are also deleterious to the catalyst activity due to increased catalyst hydrothermal deactivation. Moreover, the high concentration of water in the effluent also adds cost to downstream processing where large size equipment is necessary to separate the water from the desired light olefin products in the effluent.

Thus, a need exists for modifying an OTO reaction process or providing a new reaction process for forming light olefins, while minimizing the amount of water by-products formed in the OTO reaction process.

### SUMMARY OF THE INVENTION

The present invention provides processes for forming light olefins from methanol or from syngas through a dimethyl ether intermediate. Specifically, in one embodiment, the process is to a process for forming light olefins, wherein the process comprises the steps of: (a) contacting methanol with a first catalyst in a first reaction zone under conditions effective to convert the methanol to dimethyl ether and water; and (b) contacting the dimethyl ether with a second catalyst in a second reaction zone under conditions effective to convert the dimethyl ether to the light olefins and water. Optionally, the process further comprises the step of: (c) separating, prior to step (b), a weight majority of the dimethyl ether formed in step (a) from a weight majority of the water formed in step (a). The first catalyst optionally comprises a component selected from the group consisting of: an acidic γ-alumina, a modified zeolite, mordenite, a zeolite, ZSM-5, sulfonic acid ion exchange resin and a perfluorinated sulfonic acid ionomer. The second catalyst optionally comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof. Optionally, the first reaction zone is in a fixed bed reactor. The second reaction zone optionally is in a fluidized reactor. The methanol preferably is directed to the first reaction zone in a first feed stream, which further comprises water.

In another embodiment, the invention is to a process for forming light olefins, wherein the process comprises the steps of: (a) contacting syngas with a first catalyst in a first reaction zone under conditions effective to convert the syngas to dimethyl ether, methanol and water; and (b) contacting the dimethyl ether with a second catalyst in a second reaction zone under conditions effective to convert the dimethyl ether to the light olefins and water. Optionally, the process further comprises the step of: (c) separating, prior to step (b), a weight majority of the dimethyl ether and the methanol formed in step (a), from a weight majority of the water formed in step (a). Alternatively, the process further comprises the step of: (c) separating, prior to step (b), a weight majority of the dimethyl ether formed in step (a) from a weight majority of the methanol and water formed in step (a). Optionally, the first catalyst comprises a component selected from the group consisting of: an aluminum phosphate (AlPO₄), an acidic γ-alumina, a modified zeolite, mordenite, a zeolite, ZSM-5, sulfonic acid ion exchange resin, a perfluorinated sulfonic acid ionomer, and a copper/zinc oxide combined in a mixture or separate stages. The second catalyst optionally comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof. The first reaction zone optionally is in a fixed bed reactor, and the second reaction zone optionally is in a fluidized reactor.

In another embodiment, the invention is to a process for forming light olefins, wherein the process comprises the steps of: (a) contacting methanol with a first catalyst to form a first effluent stream comprising dimethyl ether, methanol, and water; (b) adding a recycle stream, which optionally comprises water, to the first effluent stream to form a combined stream; (c) removing water from the combined stream to form a DME concentrated stream comprising dimethyl ether and methanol; (d) contacting the dimethyl ether from the DME concentrated stream with a second catalyst to form a second effluent stream comprising the light olefins and additional water; and (e) separating the second effluent stream into a product stream and the recycle stream. Optionally, the second effluent stream comprises at least about 22 molar percent, at least about 32 molar percent, or at least about 36 molar percent light olefins, based on the total moles of light olefins and water in the second effluent stream. Step (e) optionally comprises quenching the second effluent stream under conditions effective to form an overhead stream and a bottoms stream, wherein the overhead stream comprises a weight majority of the light olefins, and the bottoms stream comprises a weight majority of the water formed in step (d), wherein the recycle stream comprises at least a portion of the bottoms stream. Alternatively, step (e) comprises: (i) compressing at least a portion of the second effluent stream to form a compressed stream; and (ii) cooling at least a portion of the compressed stream under conditions effective to form an overhead stream and a bottoms stream, wherein the overhead stream comprises a weight majority of the light olefins from the compressed stream, and the bottoms stream comprises a weight majority of the water from the compressed stream, wherein the recycle stream comprises at least a portion of the bottoms stream. In one embodiment, the first effluent stream, the combined stream and the DME concentrated stream further comprise residual methanol, and the process further comprises the step of: contacting the residual methanol in the DME concentrated stream with the second catalyst under conditions effective to convert the residual methanol to light olefins and water. Optionally, the first effluent stream, the combined stream and the DME concentrated stream further comprise residual methanol, and the process further comprises the step of: separating and recycling a weight majority of the residual methanol from the DME concentrated stream to step (a). Optionally, at least a portion of the water removed in step (c) is directed to a syngas generation unit. The first catalyst optionally comprises a component selected from the group consisting of: an acidic γ-alumina, a modified zeolite, mordenite, a zeolite, ZSM-5, sulfonic acid ion exchange resin and a perfluorinated sulfonic acid ionomer. The second catalyst optionally comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof. Step (a) optionally occurs in a fixed bed reactor, and step (d) optionally occurs in a fluidized reactor. Steps (b) and (c) optionally occur in a separation unit. Optionally, step (b) occurs outside of a separation unit, and step (c) occurs in the separation unit. The DME concentrated stream optionally comprises at least about 50, at least about 60 or at least about 70 weight percent dimethyl ether, based on the total weight of the DME concentrated stream.

In another embodiment, the invention is to a process for forming light olefins, wherein the process comprises the steps of: (a) contacting syngas and optionally recycled methanol with a first catalyst to form a first effluent stream comprising dimethyl ether, methanol and water; (b) adding a recycle stream, which optionally comprises water, to the first effluent stream to form a combined stream; (c) removing water from the combined stream to form a DME concentrated stream comprising dimethyl ether and methanol; (d) contacting the dimethyl ether from the DME concentrated stream and optionally the methanol from the DME concentrated stream with a second catalyst to form a second effluent stream comprising the light olefins and additional water; and (e) separating the second effluent stream into a product stream and the recycle stream, which is added in step (b). Optionally, the second effluent stream comprises at least about 22, at least about 32, or at least about 36 molar percent light olefins, based on the total moles of light olefins and water in the second effluent stream. The process optionally further comprises the step of: (f) separating a weight majority of the dimethyl ether in the DME concentrated stream from a weight majority of the methanol in the DME concentrated stream prior to step (d). Additionally, the process optionally further comprises the step of: (g) recycling the separated methanol from the DME concentrated stream to step (a) as the recycled methanol. Step (e) optionally comprises quenching the second effluent stream under conditions effective to form an overhead stream and a bottoms stream, wherein the overhead stream comprises a weight majority of the light olefins formed in step (d), and the bottoms stream comprises a weight majority of the water formed in step (d), wherein the recycle stream comprises at least a portion of the bottoms stream. Alternatively, step (e) comprises: (i) compressing at least a portion of the second effluent stream to form a compressed stream; and (ii) cooling at least a portion of the compressed stream under conditions effective to form an overhead stream and a bottoms stream, wherein the overhead stream comprises a weight majority of the light olefins from the compressed stream, and the bottoms stream comprises a weight majority of the water from the compressed stream, wherein the recycle stream comprises at least a portion of the bottoms stream. Optionally, at least a portion of the water removed in step (c) is directed to a syngas generation unit. The first catalyst optionally comprises a component selected from the group consisting of: an aluminum phosphate (AlPO₄), an acidic γ-alumina, a modified zeolite, mordenite, a zeolite, ZSM-5, sulfonic acid ion exchange resin, a perfluorinated sulfonic acid ionomer, and a copper/zinc oxide combined in a mixture or separate stages. The second catalyst optionally comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof. Step (a) optionally occurs in a fixed bed reactor, and step (d) optionally occurs in a fluidized reactor. In one embodiment, steps (b) and (c) occur in a separation unit. Optionally, step (b) occurs outside of a separation unit, and step (c) occurs in the separation unit. In one embodiment, the first effluent stream comprises at least about 40, at least about 50 or at least about 60 weight percent dimethyl ether, based on the total weight of the first effluent stream. The DME concentrated stream optionally comprises at least about 50, at least about 75, or at least about 85 weight percent dimethyl ether, based on the total weight of the DME concentrated stream.

In another embodiment, the invention is to a process for forming light olefins, wherein the process comprises the steps of: (a) contacting methanol with a first catalyst in a first reaction zone under conditions effective to convert the methanol to dimethyl ether and water; (b) combining the dimethyl ether, unreacted methanol, the water and a recycle stream to form a combined stream; (c) separating the combined stream into a first overhead stream and a first bottoms stream, wherein the first overhead stream comprises a weight majority of the dimethyl ether and a weight majority of the unreacted methanol from the combined stream, and the first bottoms stream comprises a weight majority of the water from the combined stream; (d) contacting the dimethyl ether and optionally the unreacted methanol in the first overhead stream with a second catalyst in a second reaction zone under conditions effective to convert the dimethyl ether and optionally the optional unreacted methanol to the light olefins and water; and (e) removing a portion of the water formed in step (d) to form the recycle stream.

In another embodiment, the invention is to a process for forming light olefins, wherein the process comprises the steps of: (a) contacting syngas and optionally methanol with a first catalyst in a first reaction zone under conditions effective to convert the syngas and optionally the methanol to dimethyl ether, methanol and water; (b) combining the dimethyl ether, the methanol, the water and a recycle stream to form a combined stream; (c) separating the combined stream into a first overhead stream and a first bottoms stream, wherein the first overhead stream comprises a weight majority of the dimethyl ether and a weight majority of the methanol from the combined stream, and the first bottoms stream comprises a weight majority of the water from the combined stream; (d) contacting the dimethyl ether and optionally the methanol in the first overhead stream with a second catalyst in a second reaction zone under conditions effective to convert the dimethyl ether and the optional methanol to the light olefins and water; and (e) removing a portion of the water formed in step (d) to form the recycle stream.

In another embodiment, the invention is to a process for debottlenecking an existing methanol to olefins reaction system, wherein the process comprises the steps of: (a) adding a methanol dehydration reactor to the existing methanol to olefins reaction system; (b) converting methanol to dimethyl ether and water in the dehydration reactor; (c) contacting the dimethyl ether with a molecular sieve catalyst composition under conditions effective to convert the dimethyl ether to light olefins and water; and (d) yielding the light olefins and water from the reaction system in an effluent stream. Optionally, the process results in at least a 10, at least a 20 or at least a 30 molar percent reduction in effluent volumetric flow rate compared to the existing methanol to olefins reaction system. The effluent stream optionally has a molar ratio of total effluent stream to light olefins contained therein of less than about 4.5, less than about 4.0 or less than about 3.5.

### BRIEF DESCRIPTION OF THE DRAWINGS

This invention will be better understood by reference to the detailed description of the invention when taken together with the attached drawings, wherein:

FIG. 1 is a flow diagram illustrating a syngas and methanol synthesis system;

FIG. 2 is a flow diagram of one embodiment of the present invention wherein methanol is converted to light olefins through a dimethyl ether intermediate;

FIG. 3 is a flow diagram of one embodiment of the present invention wherein syngas is converted to light olefins through a dimethyl ether intermediate; and

FIG. 4 is a flow diagram of one embodiment of the present invention showing a syngas or methanol to light olefins system coupled with a particularly desirable downstream processing sequence.

### DETAILED DESCRIPTION OF THE INVENTION

Introduction

The present invention provides processes for forming light olefins from methanol or from syngas through a dimethyl ether intermediate. In one embodiment, the invention is to converting a feed stream comprising methanol and/or syngas to dimethyl ether and water in the presence of a first catalyst, preferably comprising γ-alumina. If the feed stream comprises syngas, the first catalyst comprises at least two catalyst species that in combination can effect the conversion of syngas to methanol and subsequently methanol to dimethyl ether. The dimethyl ether and water preferably are separated from one another, and the separated dimethyl ether is converted to light olefins and water in the presence of a second catalyst, preferably a molecular sieve catalyst composition.

By converting the methanol and/or syngas to light olefins through a dimethyl ether intermediate and removing the water therefrom prior to converting the dimethyl ether to light olefins in an oxygenate to olefins (OTO) conversion step, the amount of water formed in the OTO conversion step is significantly less than in traditional methanol to olefin (MTO) reaction systems. As a result, the size and commensurate start-up costs associated with building an OTO reaction system according to the present invention are less than those of conventional MTO reaction systems. Further, since less water needs to be separated from the light olefin-containing effluent stream of the present invention, the start-up and operating costs associated with the separation systems utilized with the present invention are substantially less than the separation systems required for conventional MTO reaction systems.

Syngas and Methanol Synthesis Processes

As indicated above, the present invention is directed to converting syngas and/or methanol to dimethyl ether and water and converting the dimethyl ether to light olefins and water. In one embodiment, this invention is coupled with a syngas and/or methanol synthesis process, discussed in more detail hereinafter.

Generally, the production of syngas involves a reforming reaction of natural gas, mostly methane, and an oxygen source into hydrogen, carbon monoxide and/or carbon dioxide. Syngas is defined as a gas comprising carbon monoxide (CO), hydrogen (H₂) and optionally carbon dioxide (CO₂). Optionally, syngas may also include unreacted feedstocks such as methane (CH₄), ethane, propane, heavier hydrocarbons, or other compounds. Syngas production processes are well known, and include conventional steam reforming, autothermal reforming, or a combination thereof.

There are numerous technologies available for producing methanol including fermentation or the reaction of synthesis gas (syngas) derived from a hydrocarbon feed stream, which may include natural gas, petroleum liquids, carbonaceous materials including coal, recycled plastics, municipal waste or any other organic material. Methanol is typically synthesized from the catalytic reaction of syngas in a methanol synthesis reactor in the presence of a heterogeneous catalyst. For example, in one synthesis process methanol is produced using a copper/zinc oxide catalyst in a water-cooled tubular methanol reactor.

Methanol compositions can be manufactured from a hydrocarbon feed stream derived from a variety of carbon sources. Examples of such sources include biomass, natural gas, C₁-C₅ hydrocarbons, naphtha, heavy petroleum oils, or coke (i.e., coal). Preferably, the hydrocarbon feed stream comprises methane in an amount of at least about 50% by volume, more preferably at least about 70% by volume, most preferably at least about 80% by volume. In one embodiment of this invention natural gas is the preferred hydrocarbon feed source.

One way of converting the carbon source to a methanol composition is to first convert the carbon source to syngas, and then convert the syngas to the methanol composition. Any conventional process can be used. In particular, any conventional carbon oxide conversion catalyst can be used to convert the syngas to the methanol composition. In one embodiment, the carbon oxide conversion catalyst is a nickel containing catalyst.

The hydrocarbon feed stream that is used in the conversion of hydrocarbon to syngas is optionally treated to remove impurities that can cause problems in further processing of the hydrocarbon feed stream. These impurities can poison many conventional propylene and ethylene forming catalysts. A majority of the impurities that may be present can be removed in any conventional manner. The hydrocarbon feed is preferably purified to remove sulfur compounds, nitrogen compounds, particulate matter, other condensables, and/or other potential catalyst poisons prior to being converted into syngas.

In one embodiment of the invention, the hydrocarbon feed stream is passed to a syngas plant. The syngas preferably has an appropriate molar ratio of hydrogen to carbon oxide (carbon monoxide and/or carbon dioxide), as described below. The syngas plant may employ any conventional means of producing syngas, including partial oxidation, steam or CO₂ reforming, or a combination of these two chemistries.

Steam reforming generally comprises contacting a hydrocarbon with steam to form syngas. The process preferably includes the use of a catalyst.

Partial oxidation generally comprises contacting a hydrocarbon with oxygen or an oxygen-containing gas such as air to form syngas. Partial oxidation takes place with or without the use of a catalyst, although the use of a catalyst is preferred. In one embodiment, water (steam) is added with the feed in the partial oxidation process. Such an embodiment is generally referred to as autothermal reforming.

Conventional syngas-generating processes include gas phase partial oxidation, autothermal reforming, fluid bed syngas generation, catalytic partial oxidation and various processes for steam reforming.

A. Steam Reforming to Make Syngas

In the catalytic steam reforming process, hydrocarbon feeds are converted to a mixture of H₂, CO and CO₂ by reacting hydrocarbons with steam over a catalyst. This process involves the following reactions:
CH₄+H₂O⇄CO+3H₂
or
CₙHₘ+nH₂O⇄nCO+[n+(m/2)]H₂, and
CO+H₂O⇄CO₂+H₂ (shift reaction)

The reaction is carried out in the presence of a catalyst. Any conventional reforming type catalyst can be used. The catalyst used in the step of catalytic steam reforming comprises at least one active metal or metal oxide of Group 6 or Group 8-10 of the Periodic Table of the Elements. The Periodic Table of the Elements referred to herein is that from CRC Handbook of Chemistry and Physics, 82nd Edition, 2001-2002, CRC Press LLC, which is incorporated herein by reference.

In one embodiment, the catalyst contains at least one Group 6 or Group 8-10 metal, or oxide thereof, having an atomic number of 28 or greater. Specific examples of reforming catalysts that can be used are nickel, nickel oxide, cobalt oxide, chromia and molybdenum oxide. Optionally, the catalyst is employed with at least one promoter. Examples of promoters include alkali and rare earth promoters. Generally, promoted nickel oxide catalysts are preferred.

The amount of Group 6 or Group 8-10 metals in the catalyst can vary. Preferably, the catalyst includes from about 3 wt % to about 40 wt % of at least one Group 6 or Group 8-10 metal, based on total weight of the catalyst. Preferably, the catalyst includes from about 5 wt % to about 25 wt % of at least one Group 6 or Group 8-10 metal, based on total weight of the catalyst.

The reforming catalyst optionally contains one or more metals to suppress carbon deposition during steam reforming. Such metals are selected from the metals of Group 14 and Group 15 of the Periodic Table of the Elements. Preferred Group 14 and Group 15 metals include germanium, tin, lead, arsenic, antimony, and bismuth. Such metals are preferably included in the catalyst in an amount of from about 0.1 wt % to about 30 wt %, based on total weight of nickel in the catalyst.

In a catalyst comprising nickel and/or cobalt there may also be present one or more platinum group metals, which are capable of increasing the activity of the nickel and/or cobalt and of decreasing the tendency to carbon lay-down when reacting steam with hydrocarbons higher than methane. The concentration of such platinum group metal is typically in the range 0.0005 to 0.1 wt. % as metal, calculated as the whole catalyst unit. Further, the catalyst, especially in preferred forms, can contain a platinum group metal but no non-noble catalytic component. Such a catalyst is more suitable for the hydrocarbon steam reforming reaction than one containing a platinum group metal on a conventional support because a greater fraction of the active metal is accessible to the reacting gas. A typical content of platinum group metal when used alone is in the range 0.0005 to 0.5% w/w metal, calculated on the whole catalytic unit.

In one embodiment, the reformer unit includes tubes which are packed with solid catalyst granules. Preferably, the solid catalyst granules comprise nickel or other catalytic agents deposited on a suitable inert carrier material. More preferably, the catalyst is NiO supported on calcium aluminate, alumina, spinel type magnesium aluminum oxide or calcium aluminate titanate.

In yet another embodiment, both the hydrocarbon feed stream and the steam are preheated prior to entering the reformer. The hydrocarbon feedstock is preheated up to as high a temperature as is consistent with the avoiding of undesired pyrolysis or other heat deterioration. Since steam reforming is endothermic in nature, and since there are practical limits to the amount of heat that can be added by indirect heating in the reforming zones, preheating of the feed is desired to facilitate the attainment and maintenance of a suitable temperature within the reformer itself. Accordingly, it is desirable to preheat both the hydrocarbon feed and the steam to a temperature of at least 200° C.; preferably at least 400° C. The reforming reaction is generally carried out at a reformer temperature of from about 500° C. to about 1,200° C., preferably from about 800° C. to about 1,100° C., and more preferably from about 900° C. to about 1,050° C.

Gas hourly space velocity in the reformer should be sufficient for providing the desired CO to CO₂ balance in the syngas. Preferably, the gas hourly space velocity (based on wet feed) is from about 3,000 per hour to about 10,000 per hour, more preferably from about 4,000 per hour to about 9,000 per hour, and most preferably from about 5,000 per hour to about 8,000 per hour.

Any conventional reformer can be used in the step of catalytic steam reforming. The use of a tubular reformer is preferred. Preferably, the hydrocarbon feed is passed to a tubular reformer together with steam, and the hydrocarbon and steam contact a steam reforming catalyst. In one embodiment, the steam reforming catalyst is disposed in a plurality of furnace tubes that are maintained at an elevated temperature by radiant heat transfer and/or by contact with combustion gases. Fuel, such as a portion of the hydrocarbon feed, is burned in the reformer furnace to externally heat the reformer tubes therein. See, for example, Kirk-Othmer, Encyclopedia of Chemical Technology, 3rd Ed., 1990, vol. 12, p. 951; and Ullmann's Encyclopedia of Industrial Chemistry, 5th Ed., 1989, vol. A-12, p. 186, the relevant portions of each being fully incorporated herein by reference.

The ratio of steam to hydrocarbon feed will vary depending on the overall conditions in the reformer. The amount of steam employed is influenced by the requirement of avoiding carbon deposition on the catalyst, and by the acceptable methane content of the effluent at the reforming conditions maintained. On this basis, the mole ratio of steam to hydrocarbon feed in the conventional primary reformer unit is preferably from about 1.5:1 to about 5:1, preferably from about 2:1 to about 4:1.

Typically, the syngas formed comprises hydrogen and a carbon oxide. The hydrogen to carbon oxide ratio of the syngas produced will vary depending on the overall conditions of the reformer. Preferably, the molar ratio of hydrogen to carbon oxide in the syngas will range from about 1:1 to about 5:1. More preferably the molar ratio of hydrogen to carbon oxide will range from about 2:1 to about 3:1. Even more preferably the molar ratio of hydrogen to carbon oxide will range from about 2:1 to about 2.5:1. Most preferably the molar ration of hydrogen to carbon oxide will range from about 2:1 to about 2.3:1.

Steam reforming is generally carried out at superatmospheric pressure. The specific operating pressure employed is influenced by the pressure requirements of the subsequent process in which the reformed gas mixture is to be employed. Although any superatmospheric pressure can be used in practicing the invention, pressures of from about 175 psig (1,308 kPa abs.) to about 1,100 psig (7,686 kPa abs.) are desirable. Preferably, steam reforming is carried out at a pressure of from about 300 psig (2,170 kPa abs.) to about 800 psig (5,687 kPa abs.), more preferably from about 350 psig (2,515 kPa abs.) to about 700 psig (4,928 kPa abs.).

B. Partial Oxidation to Make Syngas

The invention optionally provides for the production of syngas, or CO and H₂, by oxidative conversion (also referred to herein as partial oxidation) of hydrocarbons, particularly natural gas and C₁-C₅ hydrocarbons. According to the process, one or more hydrocarbons are reacted with free-oxygen to form CO and H₂. The process is carried out with or without a catalyst. The use of a catalyst is preferred, preferably with the catalyst containing at least one non-transition or transition metal oxides. The process is essentially exothermic, and is an incomplete combustion reaction, having the following general formula:
CₙHₘ+(n/2)O₂⇄nCO+(m/2)H₂

Non-catalytic partial oxidation of hydrocarbons to H₂, CO and CO₂ is desirably used for producing syngas from heavy fuel oils, primarily in locations where natural gas or lighter hydrocarbons, including naphtha, are unavailable or uneconomical compared to the use of fuel oil or crude oil. The non-catalytic partial oxidation process is carried out by injecting preheated hydrocarbon, oxygen and steam through a burner into a closed combustion chamber. Preferably, the individual components are introduced at a burner where they meet in a diffusion flame, producing oxidation products and heat. In the combustion chamber, partial oxidation of the hydrocarbons generally occurs with less than stoichiometric oxygen at very high temperatures and pressures. Preferably, the components are preheated and pressurized to reduce reaction time. The process preferably occurs at a temperature of from about 1,350° C. to about 1,600° C., and at a pressure of from above atmospheric to about 150 atm.

Catalytic partial oxidation comprises passing a gaseous hydrocarbon mixture, and oxygen, preferably in the form of air, over reduced or unreduced composite catalysts. The reaction is optionally accompanied by the addition of water vapor (steam). When steam is added, the reaction is generally referred to as autothermal reduction. Autothermal reduction is both exothermic and endothermic as a result of adding both oxygen and water.

In the partial oxidation process, the catalyst comprises at least one transition element selected from the group consisting of Ni, Co, Pd, Ru, Rh, Ir, Pt, Os and Fe. Preferably, the catalyst comprises at least one transition element selected from the group consisting of Pd, Pt, and Rh. In another embodiment, preferably the catalyst comprises at least one transition element selected form the group consisting of Ru, Rh, and Ir.

In one embodiment, the partial oxidation catalyst further comprises at least one metal selected from the group consisting of Ti, Zr, Hf, Y, Th, U, Zn, Cd, B, Al, Ti, Si, Sn, Pb, P, Sb, Bi, Mg, Ca, Sr, Ba, Ga, V, and Sc. Also, optionally included in the partial oxidation catalyst is at least one rare earth element selected from the group consisting of La, Ce, Pr, Nd, Pm, Sm, Eu, Gd, Th, Dy, Ho, Er, Tm, Yb and Lu.

In another embodiment the catalyst employed in the process may comprise a wide range of catalytically active components, for example Pd, Pt, Rh, Ir, Os, Ru, Ni, Cr, Co, Ce, La and mixtures thereof. Materials not normally considered to be catalytically active may also be employed as catalysts, for example refractory oxides such as cordierite, mullite, mullite aluminum titanate, zirconia spinels and alumina.

In yet another embodiment, the catalyst is comprised of metals selected from those having atomic number 21 to 29, 40 to 47 and 72 to 79, the metals Sc, Ti V, Cr, Mn, Fe, Co, Ni, Cu, Zr, Nb, Mo, Tc, Ru, Rh, Pd, Ag, Hf, Ta, W, Re, Os Ir, Pt, and Au. The preferred metals are those in Group 8 of the Periodic Table of the Elements, that is Fe, Os, Co, Re, Ir, Pd, Pt, Ni, and Ru.

In another embodiment, the partial oxidation catalyst comprises at least one transition or non-transition metal deposited on a monolith support. The monolith supports are preferably impregnated with a noble metal such as Pt, Pd or Rh, or other transition metals such as Ni, Co, Cr and the like. Desirably, these monolith supports are prepared from solid refractory or ceramic materials such as alumina, zirconia, magnesia, ceria, silica, titania, mixtures thereof, and the like. Mixed refractory oxides, that is refractory oxides comprising at least two actions, may also be employed as carrier materials for the catalyst.

In one embodiment, the catalyst is retained in form of a fixed arrangement. The fixed arrangement generally comprises a fixed bed of catalyst particles. Alternatively, the fixed arrangement comprises the catalyst in the form of a monolith structure. The fixed arrangement may consist of a single monolith structure or, alternatively, may comprise a number of separate monolith structures combined to form the fixed arrangement. A preferred monolith structure comprises a ceramic foam. Suitable ceramic foams for use in the process are available commercially.

In yet another embodiment, the feed comprises methane, and the feed is injected with oxygen into the partial oxidation reformer at a methane to oxygen (i.e., O₂) ratio of from about 1.2:1 to about 10:1. Preferably the feed and oxygen are injected into the reformer at a methane to oxygen ratio of from about 1.6:1 to about 8:1, more preferably from about 1.8:1 to about 4:1.

Water may or may not be added to the partial oxidation process. When added, the concentration of water injected into the reformer is not generally greater than about 65 mole %, based on total hydrocarbon and water feed content. Preferably, when water is added, it is added at a water to methane ratio of not greater than 3:1, preferably not greater than 2:1.

The catalyst may or may not be reduced before the catalytic reaction. In one embodiment, the catalyst is reduced and reduction is carried out by passing a gaseous mixture comprising hydrogen and inert gas (e.g., N₂, He, or Ar) over the catalyst in a fixed bed reactor at a catalyst reduction pressure of from about 1 atm to about 5 atm, and a catalyst reduction temperature of from about 300° C. to about 700° C. Hydrogen gas is used as a reduction gas, preferably at a concentration of from about 1 mole % to about 100 mole %, based on total amount of reduction gas. Desirably, the reduction is further carried out at a space velocity of reducing gas mixture of from about 103 cm³/g·hr to about 105 cm³/g·hr for a period of from about 0.5 hour to about 20 hours.

In one embodiment, the partial oxidation catalyst is not reduced by hydrogen. When the catalyst is not reduced by hydrogen before the catalytic reaction, the reduction of the catalyst can be effected by passing the hydrocarbon feed and oxygen (or air) over the catalyst at temperature in the range of from about 500° C. to about 900° C. for a period of from about 0.1 hour to about 10 hours.

In the partial oxidation process, carbon monoxide (CO) and hydrogen (H₂) are formed as major products, and water and carbon dioxide (CO₂) as minor products. The gaseous product stream comprises the above mentioned products, unconverted reactants (i.e. methane or natural gas and oxygen) and components of feed other than reactants.

When water is added in the feed, the H₂:CO mole ratio in the product is increased by the shift reaction: CO+H₂O⇄H₂+CO₂. This reaction occurs simultaneously with the oxidative conversion of the hydrocarbon in the feed to CO and H₂ or syngas. The hydrocarbon used as feed in the partial oxidation process is preferably in the gaseous phase when contacting the catalyst. The partial oxidation process is particularly suitable for the partial oxidation of methane, natural gas, associated gas or other sources of light hydrocarbons. In this respect, the term “light hydrocarbons” is a reference to hydrocarbons having from 1 to 5 carbon atoms. The process may be advantageously applied in the conversion of gas from naturally occurring reserves of methane which contain substantial amounts of carbon dioxide. In one embodiment, the hydrocarbon feed preferably contains from about 10 mole % to about 90 mole % methane, based on total feed content. More preferably, the hydrocarbon feed contains from about 20 mole % to about 80 mole % methane, based on total feed content. In another embodiment, the feed comprises methane in an amount of at least 50% by volume, more preferably at least 70% by volume, and most preferably at least 80% by volume.

In one embodiment of the invention, the hydrocarbon feedstock is contacted with the catalyst in a mixture with an oxygen-containing gas. Air is suitable for use as the oxygen-containing gas. Substantially pure oxygen as the oxygen-containing gas is preferred on occasions where there is a need to avoid handling large amounts of inert gas such as nitrogen. The feed optionally comprises steam.

In another embodiment of the invention, the hydrocarbon feedstock and the oxygen-containing gas are preferably present in the feed in such amounts as to give an oxygen-to-carbon ratio in the range of from about 0.3:1 to about 0.8:1, more preferably, in the range of from about 0.45:1 to about 0.75:1. References herein to the oxygen-to-carbon ratio refer to the ratio of oxygen in the from of oxygen molecules (O₂) to carbon atoms present in the hydrocarbon feedstock. Preferably, the oxygen-to-carbon ratio is in the range of from about 0.45:1 to about 0.65:1, with oxygen-to-carbon ratios in the region of the stoichiometric ratio of 0.5:1, that is ratios in the range of from about 0.45:1 to about 0.65:1, being more preferred. When steam is present in the feed, the steam-to-carbon ratio is not greater than about 3.0:1, more preferably not greater than about 2.0:1. The hydrocarbon feedstock, the oxygen-containing gas and steam, if present, are preferably well mixed prior to being contacted with the catalyst.

The partial oxidation process is operable over a wide range of pressures. For applications on a commercial scale, elevated pressures, that is pressures significantly above atmospheric pressure, are preferred. In one embodiment, the partial oxidation process is operated at pressures of greater than atmospheric up to about 150 bars. Preferably, the partial oxidation process is operated at a pressure in the range of from about 2 bars to about 125 bars, more preferably from about 5 bars to about 100 bars.

The partial oxidation process is also operable over a wide range of temperatures. At commercial scale, the feed is preferably contacted with the catalyst at high temperatures. In one embodiment, the feed mixture is contacted with the catalyst at a temperature in excess of 600° C. Preferably, the feed mixture is contacted with the catalyst at a temperature in the range of from about 600° C. to about 1,700° C., more preferably from about 800° C. to about 1,600° C. The feed mixture is preferably preheated prior to contacting the catalyst.

The feed is provided during the operation of the process at a suitable space velocity to form a substantial amount of CO in the product. In one embodiment, gas space velocities (expressed in normal liters of gas per kilogram of catalyst per hour) are in the range of from about 20,000 Nl/kg/hr to about 100,000,000 Nl/kg/hr, more preferably in the range of from about 50,000 Nl/kg/hr to about 50,000,000 Nl/kg/hr, and most preferably in the range of from about 500,000 Nl/kg/hr to about 30,000,000 Nl/kg/hr.

C. Combination Syngas Synthesis Processes

Combination reforming processes can also be incorporated into this invention. Examples of combination reforming processes include autothermal reforming and fixed bed syngas generation. These processes involve a combination of gas phase partial oxidation and steam reforming chemistry.

The autothermal reforming process preferably comprises two syngas generating processes, a primary oxidation process and a secondary steam reforming process. In one embodiment, a hydrocarbon feed stream is steam reformed in a tubular primary reformer by contacting the hydrocarbon and steam with a reforming catalyst to form a hydrogen and carbon monoxide containing primary reformed gas, the carbon monoxide content of which is further increased in the secondary reformer. In one embodiment, the secondary reformer includes a cylindrical refractory lined vessel with a gas mixer, preferably in the form of a burner in the inlet portion of the vessel and a bed of nickel catalyst in the lower portion. In a more preferred embodiment, the exit gas from the primary reformer is mixed with air and residual hydrocarbons, and the mixed gas partial oxidized to carbon monoxides.

In another embodiment incorporating the autothermal reforming process, partial oxidation is carried out as the primary oxidating process. Preferably, hydrocarbon feed, oxygen, and optionally steam, are heated and mixed at an outlet of a single large coaxial burner or injector which discharges into a gas phase partial oxidation zone. Oxygen is preferably supplied in an amount which is less than the amount required for complete combustion.

Upon reaction in the partial oxidation combustion zone, the gases flow from the primary reforming process into the secondary reforming process. In one embodiment, the gases are passed over a bed of steam reforming catalyst particles or a monolithic body, to complete steam reforming. Desirably, the entire hydrocarbon conversion is completed by a single reactor aided by internal combustion.

In an alternative embodiment of the invention, a fixed bed syngas generation process is used to form syngas. In the fixed bed syngas generation process, hydrocarbon feed and oxygen or an oxygen-containing gas are introduced separately into a fluid catalyst bed. Preferably, the catalyst is comprised of nickel and supported primarily on alpha alumina.

The fixed bed syngas generation process is carried out at conditions of elevated temperatures and pressures that favor the formation of hydrogen and carbon monoxide when, for example, methane is reacted with oxygen and steam. Preferably, temperatures are in excess of about 1,700° F. (927° C.), but not so high as to cause disintegration of the catalyst or the sticking of catalyst particles together. Preferably, temperatures range from about 1,750° F. (954° C.) to about 1,950° F. (1,066° C.), more preferably, from about 1,800° F. (982° C.) to about 1,850° F. (1,010° C.).

Pressure in the fixed bed syngas generation process may range from atmospheric to about 40 atmospheres. In one embodiment, pressures of from about 20 atmospheres to about 30 atmospheres are preferred, which allows subsequent processes to proceed without intermediate compression of product gases.

In one embodiment of the invention, methane, steam, and oxygen are introduced into a fluid bed by separately injecting the methane and oxygen into the bed. Alternatively, each stream is diluted with steam as it enters the bed. Preferably, methane and steam are mixed at a methane to steam molar ratio of from about 1:1 to about 3:1, and more preferably from about 1.5:1 to about 2.5:1, and the methane and steam mixture is injected into the bed. Preferably, the molar ratio of oxygen to methane is from about 0.2:1 to about 1.0:1, more preferably from about 0.4:1 to about 0.6:1.

In another embodiment of the invention, the fluid bed process is used with a nickel based catalyst supported on alpha alumina. In another embodiment, silica is included in the support. The support is preferably comprised of at least 95 wt % alpha alumina, more preferably at least about 98% alpha alumina, based on total weight of the support.

In one embodiment, a gaseous mixture of hydrocarbon feedstock and oxygen-containing gas are contacted with a reforming catalyst under adiabatic conditions. For the purposes of this invention, the term “adiabatic” refers to reaction conditions in which substantially all heat loss and radiation from the reaction zone are prevented, with the exception of heat leaving in the gaseous effluent stream of the reactor.

D. Converting Syngas to Methanol

In one embodiment, syngas is sent to a methanol synthesis process and is converted to methanol. The methanol synthesis process is accomplished in the presence of a methanol synthesis catalyst.

In one embodiment, the syngas is sent as is to the methanol synthesis process. In another embodiment, the hydrogen, carbon monoxide, and/or carbon dioxide content of the syngas is adjusted for efficiency of conversion. Desirably, the syngas input to the methanol synthesis reactor has a molar ratio of hydrogen (H₂) to carbon oxides (CO+CO₂) in the range of from about 0.5:1 to about 20:1, preferably in the range of from about 2:1 to about 10:1. In another embodiment, the syngas has a molar ratio of hydrogen (H₂) to carbon monoxide (CO) of at least 2:1. Carbon dioxide is optionally present in an amount of not greater than 50% by weight, based on total weight of the syngas.

Desirably, the stoichiometric molar ratio is sufficiently high so as maintain a high yield of methanol, but not so high as to reduce the volume productivity of methanol. Preferably, the syngas fed to the methanol synthesis has a stoichiometric molar ratio (i.e., a molar ratio of H₂:(2CO+3CO₂)) of from about 1.0:1 to about 2.7:1, more preferably from about 1.1 to about 2.0, more preferably a stoichiometric molar ratio of from about 1.2:1 to about 1.8:1.

The CO₂ content, relative to that of CO, in the syngas should be high enough so as to maintain an appropriately high reaction temperature and to minimize the amount of undesirable by-products such as paraffins. At the same time, the relative CO₂ to CO content should not be too high so as to reduce methanol yield. Desirably, the syngas contains CO₂ and CO at a molar ratio of from about 0.5 to about 1.2, preferably from about 0.6 to about 1.0.

In one embodiment, the catalyst used in the methanol synthesis process includes an oxide of at least one element selected from the group consisting of copper, silver, zinc, boron, magnesium, aluminum, vanadium, chromium, manganese, gallium, palladium, osmium and zirconium. Preferably, the catalyst is a copper and zinc based catalyst, more preferably in the form of copper, copper oxide, and zinc oxide.

In another embodiment, the catalyst used in the methanol synthesis process is a copper based catalyst, which includes an oxide of at least one element selected from the group consisting of silver, zinc, boron, magnesium, aluminum, vanadium, chromium, manganese, gallium, palladium, osmium and zirconium. Preferably, the catalyst contains copper oxide and an oxide of at least one element selected from the group consisting of zinc, magnesium, aluminum, chromium, and zirconium. In one embodiment, the methanol synthesis catalyst is selected from the group consisting of: copper oxides, zinc oxides and aluminum oxides. More preferably, the catalyst contains oxides of copper and zinc.

In yet another embodiment, the methanol synthesis catalyst comprises copper oxide, zinc oxide, and at least one other oxide. Preferably, the at least one other oxide is selected from the group consisting of zirconium oxide, chromium oxide, vanadium oxide, magnesium oxide, aluminum oxide, titanium oxide, hafnium oxide, molybdenum oxide, tungsten oxide, and manganese oxide.

In various embodiments, the methanol synthesis catalyst comprises from about 10 wt % to about 70 wt % copper oxide, based on total weight of the catalyst. Preferably, the methanol synthesis contains from about 15 wt % to about 68 wt % copper oxide, and more preferably from about 20 wt % to about 65 wt % copper oxide, based on total weight of the catalyst.

In one embodiment, the methanol synthesis catalyst comprises from about 3 wt % to about 30 wt % zinc oxide, based on total weight of the catalyst. Preferably, the methanol synthesis catalyst comprises from about 4 wt % to about 27 wt % zinc oxide, more preferably from about 5 wt % to about 24 wt % zinc oxide.

In embodiments in which copper oxide and zinc oxide are both present in the methanol synthesis catalyst, the ratio of copper oxide to zinc oxide can vary over a wide range. Preferably in such embodiments, the methanol synthesis catalyst comprises copper oxide and zinc oxide in a Cu:Zn atomic ratio of from about 0.5:1 to about 20:1, preferably from about 0.7:1 to about 15:1, more preferably from about 0.8:1 to about 5:1.

The methanol synthesis catalyst is made according to conventional processes. Examples of such processes can be found in U.S. Pat. Nos. 6,114,279; 6,054,497; 5,767,039; 5,045,520; 5,254,520; 5,610,202; 4,666,945; 4,455,394; 4,565,803; 5,385,949, with the descriptions of each being fully incorporated herein by reference.

In one embodiment, the syngas formed in the syngas conversion plant is cooled prior to being sent to the methanol synthesis reactor. Preferably, the syngas is cooled so as to condense at least a portion of the water vapor formed during the syngas process.

The methanol synthesis process implemented in the present invention can be any conventional methanol synthesis process. Examples of such processes include batch processes and continuous processes. Continuous processes are preferred. Tubular bed processes and fluidized bed processes are particularly preferred types of continuous processes.

In general, the methanol synthesis process takes place according to the following reactions:
CO+2H₂→CH₃OH
CO₂+3H₂→CH₃OH+H₂O

The methanol synthesis process is effective over a wide range of temperatures. In one embodiment, the syngas is contacted with the methanol synthesis catalyst at a temperature in the range of from about 302° F. (150° C.) to about 842° F. (450° C.), preferably in a range of from about 347° F. (175° C.) to about 662° F. (350° C.), more preferably in a range of from about 392° F. (200° C.) to about 572° F. (300° C.).

The process is also operable over a wide range of pressures. In one embodiment, the syngas is contacted with the methanol synthesis catalyst at a pressure in the range of from about 15 atmospheres to about 125 atmospheres, preferably in a range of from about 20 atmospheres to about 100 atmospheres, more preferably in a range of from about 25 atmospheres to about 75 atmospheres.

Gas hourly space velocities vary depending upon the type of continuous process that is used. Desirably, gas hourly space velocity of flow of gas through the catalyst bed is in the range of from about 50 hr⁻¹ to about 50,000 hr⁻¹. Preferably, gas hourly space velocity of flow of gas through the catalyst bed is in the range of from about 250 hr⁻¹ to about 25,000 hr⁻¹, more preferably from about 500 hr⁻¹ to about 10,000 hr⁻¹.

The methanol synthesis process produces a variety of hydrocarbons as by-products. According to the methanol composition of this invention, it is desirable to operate the process so as to maximize not only the amount of methanol formed, but also aldehydes and other alcohols which are particularly desirable in the conversion of oxygenates to olefins. In is particularly appropriate to maximize the amount of methanol formed in the methanol synthesis, and remove hydrocarbons less desirable in the conversion of oxygenates to olefins from the crude methanol product stream formed in the methanol synthesis reactor.

E. Refining Crude Methanol to Make Methanol Product

In conventional methanol synthesis systems, the crude methanol product mixture formed in the methanol synthesis unit is further processed after reaction to obtain a desirable methanol-containing composition. Processing is accomplished by any conventional means. Examples of such means include distillation, selective condensation, and selective adsorption. Process conditions, e.g., temperatures and pressures, can vary according to the particular methanol composition desired. It is particularly desirable to minimize the amount of water and light boiling point components in the methanol-containing composition, but without substantially reducing the amount of methanol and desirable aldehydes and/or other desirable alcohols also present.

In one processing system, the crude methanol product from the methanol synthesis reactor is sent to a let down vessel so as to reduce the pressure to about atmospheric or slightly higher. This let down in pressure allows undesirable light boiling point components to be removed from the methanol composition as a vapor. The vapor is desirably of sufficient quality to use a fuel.

In another processing system, the crude methanol is sent from the methanol synthesizing unit to a distillation system. The distillation system contains one or more distillation columns which are used to separate the desired methanol composition from water and hydrocarbon by-products. Desirably, the methanol composition that is separated from the crude methanol comprises a majority of the methanol and a majority of aldehyde and/or alcohol supplements contained in the crude alcohol prior to separation. Preferably, the methanol composition that is separated from the crude methanol comprises a majority of the acetaldehyde and/or ethanol, if any, contained in the crude methanol prior to separation.

The distillation system optionally includes a step of treating the methanol stream being distilled so as to remove or neutralize acids in the stream. Preferably, a base is added in the system that is effective in neutralizing organic acids that are found in the methanol stream. Conventional base compounds can be used. Examples of base compounds include alkali metal hydroxide or carbonate compounds, and amine or ammonium hydroxide compounds. In one particular embodiment, about 20 ppm to about 120 ppm w/w of a base composition, calculated as stoichiometrically equivalent NaOH, is added, preferably about 25 ppm to about 100 ppm w/w of a base composition, calculated as stoichiometrically equivalent NaOH, is added.

The invention can include any distillation system that produces a “fusel oil” stream, which includes C1-C4 alcohols, ketones, aldehydes and water. The fusel oil stream has a boiling point higher than that of methanol. It is especially advantageous when the fusel oil stream is liquid taken from a column fed with the crude methanol from the let-down vessel or with the bottoms liquid from a column fed with such crude methanol, the off-take point being at a level below the feed level. Alternatively or additionally, the fusel oil stream is taken from a level above the feed level in such a column. Optionally, the distillation system is operated to recover the C₂-C₄ alcohols along with the methanol rather than in the fusel oil stream.

Examples of distillation systems include the use of single and two column distillation columns. Preferably, the single column embodiment operates to remove volatiles in the overhead, methanol product in a relatively high side draw stream, fusel oil as vapor above the feed introduction point (but below the methanol side draw stream) and/or as liquid below the feed introduction point, and water as a bottoms stream.

In one embodiment of a two column system, the first column is a “topping column” from which volatiles or “light ends” are taken overhead and methanol-containing liquid as bottoms. A non-limiting list of possible light ends includes hydrogen, carbon monoxide and methane. The second column is a “refining column” from which methanol product is taken as an overhead stream or as a relatively high side draw stream, and water is removed as a bottoms stream. In this embodiment, the refining column includes at least one side draw stream for fusel oil as vapor above the feed and/or as liquid below the feed.

In another embodiment of a two column system, the first column is a water-extractive column in which there is a water feed introduced at a level above the crude methanol feed level. It is desirable to feed sufficient water to produce a bottoms liquid containing over 40% w/w water, preferably 40% to 60% w/w water, and more preferably 80% to 95% w/w water. This column optionally includes one or more direct fusel oil side off-takes.

In yet another embodiment, the distillation system is one in which an aqueous, semi-crude methanol is taken as liquid above the feed in a single or refining column. The semi-crude methanol is passed to a refining column, from which methanol product is taken overhead or as a relatively high side draw stream. Preferably, water or aqueous methanol is taken as a bottoms stream. Alternatively, undesirable by-products are removed from the crude methanol stream from the methanol synthesis reactor by adsorption. In such a system, fusel oil can be recovered by regenerating the adsorbent.

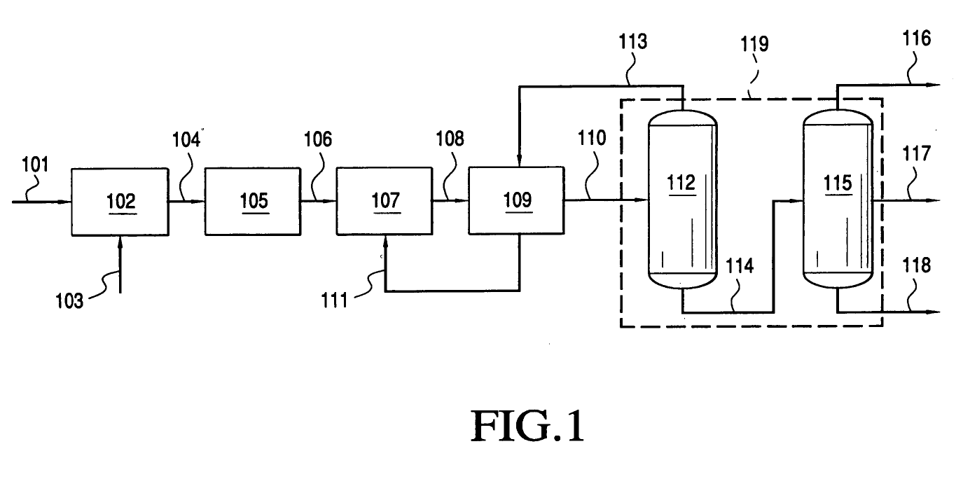

An exemplary methanol synthesis system is illustrated in FIG. 1 and will now be described in greater detail. As shown in FIG. 1, a feed stream 101, which preferably includes natural gas, is directed to a desulfurization zone 102. Prior to entering the desulfurization zone 102, the feed stream 101 optionally is compressed by one or more compressors, not shown, to facilitate movement of the feed stream 101 and various intermediate streams through the methanol synthesis system. In one embodiment, desulfurization zone 102 comprises a hydrotreating zone, an adsorption zone and a saturation zone. The natural gas from feed stream 101 contacts hydrogen under pressure in the hydrotreating zone under conditions effective to convert any sulfur-containing components contained therein into H₂S. The H₂S is then chemically adsorbed in the adsorption zone onto a ZnO adsorbent. In this manner, the desulfurization zone 102 is able to remove sulfur compounds to a sufficient level to minimize deactivation of the reforming catalyst.

In one embodiment, water from water stream 103 increases the water content of, and more preferably saturates, the feed stream 101 after the sulfur-containing components have been removed therefrom, as discussed above. In one embodiment, water contacts the desulfurized stream under conditions effective to saturate the desulfurized stream or increase the water content thereof. For example, the saturization zone may include a packed or tray column wherein water contacts the desulfurized stream in a countercurrent manner under conditions effective to saturate or increase the water content of the desulfurized stream. Ultimately, desulfurized feed stream 104 is yielded from the desulfurization zone 102 and directed to a reforming unit 105. Saturation of the feed stream 101 and/or desulfurized stream 104 is particularly beneficial if the reforming unit 105 implements a steam reforming process as a water-containing or saturated desulfurized feed stream 104 may be necessary in order for the steam reforming process to convert the desulfurized feed stream 104 to syngas in syngas stream 106. Preferably, desulfurized feed stream 104 comprises less than 5 weight percent, more preferably less than 1 weight percent, and most preferably less than 0.01 weight percent sulfur-containing compounds, based on the total weight of the saturated desulfurized feed stream 104.

The reforming unit 105 converts the natural gas in saturated desulfurized feed stream 104 to syngas in syngas stream 106. Generally, the production of syngas involves a combustion reaction of natural gas, mostly methane, and an oxygen source, e.g., air, into hydrogen, carbon monoxide and/or carbon dioxide. Syngas production processes are well known, and include conventional steam reforming, autothermal reforming, or a combination thereof. Thus, reforming unit 105 may be a steam reforming unit, a partial oxidation unit, an autothermal reforming unit, and/or a combined reforming unit, e.g., a unit that combines two or more of these reforming processes. Optionally, water is injected directly into the reforming unit 105, particularly if the reforming unit 105 provides a steam reforming process. Resulting syngas stream 106 is directed to a compression zone 107, wherein the syngas stream 106 is compressed in one or more stages to form compressed stream 108. Preferably, the compression zone 107 includes one or more centrifugal compressors. Compressed stream 108 is then directed to a methanol synthesis unit 109, wherein the syngas in compressed stream 108 contacts a methanol synthesis catalyst under conditions effective to convert at least a portion of the syngas to crude methanol in crude methanol stream 110. Optionally, unreacted syngas from methanol synthesis unit 109 is recycled to compression zone 107 as shown by unreacted syngas stream 111.

The crude methanol in crude methanol stream 110 includes light ends, methanol, water, and fusel oil. Preferably, prior to introduction into separation zone 119, the crude methanol stream 110 is treated with a caustic medium, not shown, in a caustic wash unit, not shown, under conditions effective to increase the pH of the crude methanol stream 110. As a result, the crude methanol stream 110 also optionally includes dissolved caustic salts. As shown, crude methanol stream 110 is directed to a separation zone 119, which is adapted to separate one or more of these components and isolate a relatively pure methanol stream. The separation zone 119 includes a light ends separation unit 112, such as a topping column, and a refining column 115. Crude methanol stream 110 is first directed to the light ends separation unit 112, wherein conditions are effective to separate the crude methanol stream 110 into light ends stream 113 and bottoms crude methanol stream 114, which contains methanol, water, fusel oil, and optionally dissolved caustic salts. At least a portion of the light ends stream 113 preferably is recycled to methanol synthesis unit 109, as shown, for further conversion to methanol while the bottoms crude methanol stream 114 is directed to refining column 115 for further processing. In refining column 115, the bottoms crude methanol stream 114 is subjected to conditions effective to separate the bottoms crude methanol stream 114 into a refined methanol stream 116, a fusel oil stream 117, and a water stream 118. A majority of the caustic salts, if any, from bottoms crude methanol stream 114 are dissolved in water stream 118. Preferably, refined methanol stream 116 contains at least 90 weight percent, more preferably at least 95 weight percent and most preferably at least 99 weight percent methanol, based on the total weight of the refined methanol stream 116. Preferably, refined methanol stream 116 contains less than 5.0 weight percent, more preferably less than 1.0 weight percent and most preferably less than 0.25 weight percent water, based on the total weight of the refined methanol stream 116.

Converting Methanol and/or Syngas to Dimethyl Ether

In one embodiment, the present invention is directed to a process for forming light olefins from methanol and/or syngas through a dimethyl ether intermediate. Specifically, the process includes: (a) contacting methanol and/or syngas with a first catalyst in a first reaction zone under conditions effective to convert the methanol and/or syngas to dimethyl ether and water; and (b) contacting the dimethyl ether with a second catalyst in a second reaction zone under conditions effective to convert the dimethyl ether to the light olefins and water. As used herein, the terms “first reaction step” and “first step” refer to the conversion of methanol and/or syngas to dimethyl ether and water, and the terms “second reaction step” and “second step” mean the conversion of the dimethyl ether to light olefins and water.

In this embodiment of the present invention, a first feed stream or first feedstock is directed to the first reaction zone. The first feed stream comprises the methanol, the syngas or both the methanol and the syngas. In one embodiment, the first feed stream also comprises one or more additional components such as, but not limited to, water, nitrogen, methane, ethane, propane, ethylene, propylene, or other oxygenates such as alcohols, ethers, aldehydes, and ketones.

In one embodiment, the first feed stream comprises at least about 5 weight percent methanol, more preferably at least about 50 weight percent methanol, and most preferably at least about 90 weight percent methanol, based on the total weight of the first feed stream. Additionally or alternatively, the first feed stream comprises at least about 5 weight percent syngas, preferably at least about 50 weight percent syngas, and most preferably at least about 90 weight percent syngas, based on the total weight of the first feed stream. If the first feed stream comprises both methanol and syngas, then the molar ratio of methanol to syngas in the first feed stream is preferably less than 1.0, more preferably less than 0.5, and most preferably less than 0.1. For the purpose of these ratios, a mole of syngas comprises two mole of H₂ and one mole of CO. In this embodiment, the total amount of methanol and syngas, collectively, in the first feed stream preferably is at least about 50 weight percent, more preferably at least about 80 weight percent and most preferably at least about 95 weight percent, based on the total weight of the first feed stream.

If the first feed stream comprises water, then the first feed stream preferably comprises less than 50 weight percent water, more preferably less than 10 weight percent water, and most preferably less than 1 weight percent water, based on the total weight of the first feed stream. If the first feed stream comprises CO₂, then the first feed stream preferably comprises less than 50 weight percent CO₂, more preferably less than 30 weight percent CO₂, more preferably less than 20 weight percent CO₂, and most preferably less than 10 weight percent CO₂, based on the total weight of the first feed stream. Other possible components at less than 10 wt % of each include nitrogen, methane, ethane, propane, ethylene, propylene, or other oxygenates such as alcohols, ethers, aldehydes, and ketones.

The first step of converting the methanol and/or syngas from the first feed stream to dimethyl ether and water may occur in a variety of different types of reaction vessels. In one embodiment, the first reaction zone is in a fixed bed reactor. Alternatively, the first reaction zone is in a fluidized reactor, a moving bed reactor, a tubular reactor, and a radial flow reactor.

The reaction conditions in the first reaction zone may vary widely depending on the type of reactor used and the composition of the first feed stream. Preferably, the temperature in the first reaction zone ranges from about 150° C. to about 450° C. and most preferably from about 175° C. to about 350° C. The pressure in the first reaction zone preferably ranges from about 1,500 kPaa to about 12,000 kPaa, more preferably from about 2,000 kPaa to about 10,000 kPaa, and most preferably from about 2,500 kPaa to about 7,500 kPaa. If the first reaction zone is a fluidized system, then the first reaction zone preferably has a gas superficial velocity (GSV) of from about 0.1 m/s to about 30 m/s, more preferably from about 0.5 m/s to about 25 m/s, and most preferably from about 3 m/s to about 15 m/s. The first reaction zone in this embodiment preferably has a weight hourly space velocity (WHSV) of from about 1 hr⁻¹ to about 500 hr⁻¹, more preferably from about 2 hr⁻¹ to about 200 hr⁻¹, and most preferably from about 5 hr⁻¹ to about 100 hr^{-1.}

As indicated above, in the first reaction zone, the methanol in the first feed stream preferably contacts a first catalyst under conditions effective to convert the methanol to dimethyl ether and water. Additionally or alternatively, syngas in the first feed stream contacts a first catalyst under conditions effective to convert the syngas to methanol and subsequently the methanol to dimethyl ether and water. Particularly if the first feed stream comprises methanol, the first catalyst preferably comprises as one of its components a dehydration catalyst. Optionally, the first catalyst comprises a component selected from the group consisting of: an acidic alumina, a modified zeolite, mordenite, a zeolite, ZSM-5, sulfonic acid ion exchange resin and a perfluorinated sulfonic acid ionomer.

As indicated above, the first feed stream optionally comprises syngas. The first catalyst used to convert the syngas to dimethyl ether may vary widely. In one embodiment, the first catalyst comprises a mixture of methanol synthesis catalyst and one or more of the dehydration catalysts listed above. Alternatively, the first catalyst comprises a catalyst composition having a syngas to methanol conversion site in addition to a methanol dehydration site. Thus, without limiting the invention to any particular reaction mechanism, the syngas in the first feed stream may be converted to dimethyl ether through a methanol intermediate. Most preferably, the first catalyst in this embodiment preferably comprises a dehydration catalyst component selected from the group consisting of: an aluminum phosphate, an acidic γ-alumina, a modified zeolite, mordenite, a zeolite, ZSM-5, sulfonic acid ion exchange resin, a perfluorinated sulfonic acid ionomer, and a methanol synthesis catalyst component such as copper/zinc oxide or other methanol synthesis catalyst discussed earlier combined in a mixture or separate stages within the first reaction zone.

The dimethyl ether and water formed in the first step preferably are yielded from the first reaction zone in a first effluent stream. Thus, the first effluent stream comprises dimethyl ether and water. Typically, the first effluent stream comprises DME, water, residual methanol, and optionally unreacted residual syngas (if the first feed stream comprised syngas). Preferably, the first effluent stream comprises at least about 40, more preferably at least about 50, and most preferably at least about 60 weight percent dimethyl ether, based on the total weight of the first effluent stream. The first effluent stream also will comprise water, typically at least about 5, at least about 10 or at least about 15 weight percent water, based on the total weight of the first effluent stream. The first effluent stream preferably comprises less than about 40, or preferably less than about 30, and most preferably less than about 20 weight percent water, based on the total weight of the first effluent stream. Additionally, the first effluent stream may comprise one or more additional components such as, but not limited to, methanol, carbon monoxide, carbon dioxide, hydrogen, nitrogen, methane, ethane, propane, ethylene, propylene, or other oxygenates such as alcohols, ethers, aldehydes, and ketones. If the first effluent stream comprises methanol, for example unreacted residual methanol that has passed through the first reaction zone, then the first effluent stream preferably comprises less than about 40 weight percent methanol, more preferably less than 30 weight percent methanol, and most preferably less than 20 weight percent methanol, based on the total weight of the first effluent stream. In terms of lower range limits, the first effluent stream optionally comprises at least about 5, at least about 10 or at least about 15 weight percent methanol, based on the total weight of the first effluent stream.

Preferably, prior to the second reaction step, a weight majority of the dimethyl ether formed in the first reaction step is separated from a weight majority of the water formed in the first reaction step. This separation step optionally is achieved by distilling the dimethyl ether from the water in one or more distillation columns based on the different volatilities of dimethyl ether and water. It is contemplated, however, that other separation techniques such as single or multiple series of flash separators or adsorbent beds may be used to separate the dimethyl ether from the water formed in the first reaction step. By removing the water from the dimethyl ether formed in the first reaction step, and directing the separated dimethyl ether to the second reaction zone for conversion to light olefins, the total volumetric flow rate of reactants sent to the second reaction step as well as the total volumetric flow rate of products formed in the second reaction step can be minimized without a decrease in overall light olefins production. As a result, the size of the second reaction zone and the disengaging zone associated therewith can be minimized resulting in a significant commercial savings.

Specifically, in one embodiment of the present invention, the first effluent stream is separated into a DME concentrated stream and a water concentrated stream. The DME concentrated stream is also referred to herein as the “first overhead stream,” and the water concentrated stream is also referred to herein as the “first bottoms stream.” The DME concentrated stream preferably comprises at least about 50 weight percent DME, more preferably at least about 75 weight percent DME, and most preferably at least about 85 weight percent DME, based on the total weight of the DME concentrated stream. The DME concentrated stream preferably comprises less than about 5 weight percent water, more preferably less than about 1 weight percent water, and most preferably less than about 0.1 weight percent water, based on the total weight of the DME concentrated stream. The water concentrated stream preferably comprises at least about 80 weight percent water, optionally at least about 90 weight percent water, and optionally at least about 99 weight percent water, based on the total weight of the water concentrated stream. The water concentrated stream preferably comprises less than about 5 weight percent DME, more preferably less than about 1 weight percent DME, and most preferably less than about 0.1 weight percent DME, based on the total weight of the water concentrated stream.

If the first effluent stream comprises residual syngas, then the first effluent stream preferably is cooled to a point to allow separation of the residual syngas from the remainder of the first effluent stream. The unreacted syngas ideally is recycled to relative extinction to the first reaction zone for further conversion thereof to methanol and dimethyl ether.

The methanol from the first effluent stream may be separated into the DME concentrated stream and/or the water concentrated stream. In one preferred embodiment, a weight majority of the methanol and DME from the first effluent stream is separated into the DME concentrated stream. Thus, the DME concentrated stream optionally comprises at least about 5 weight percent methanol, preferably about 10 weight percent methanol, and most preferably at least about 15 weight percent methanol, based on the total weight of the DME concentrated stream. As a result, in one embodiment the process further comprises the step of separating, prior to the second reaction step, a weight majority of the dimethyl ether and the methanol formed in the first reaction step, from a weight majority of the water formed in the first reaction step.

It is contemplated, however, that some or a majority of the methanol from the first effluent stream may be separated into the water concentrated stream. Thus, in this embodiment, the process further comprises the step of separating, prior to the second reaction step, a weight majority of the dimethyl ether formed in the first reaction step from a weight majority of the methanol and water formed in the first reaction step. In this embodiment, the water concentrated stream optionally comprises at least about 10, at least about 20 or at least about 30 weight percent methanol, based on the total weight of the water concentrated stream. Optionally, the water concentrated stream comprises less than about 50 weight percent methanol, less than about 40 weight percent methanol, or less than about 30 weight percent methanol, based on the total weight of the water concentrated stream.

It is further contemplated that the methanol may be separated from the DME and from the water, optionally through a side draw stream and/or with a plurality of separation units. In this embodiment, the first effluent stream is separated into at least three derivative streams. Specifically, the first effluent stream is separated into a DME concentrated stream, a methanol concentrated stream and a water concentrated stream. In this embodiment, the DME concentrated stream preferably comprises at least about 80 weight percent DME, more preferably at least about 90 weight percent DME, and most preferably at least about 99 weight percent DME, based on the total weight of the DME concentrated steam. In this embodiment, the DME concentrated stream preferably comprises less than about 20 weight percent methanol, more preferably less than about 10 weight percent methanol, and most preferably less than about 1 weight percent methanol, based on the total weight of the DME concentrated stream. The DME concentrated stream in this embodiment also preferably comprises less than about 5 weight percent water, more preferably less than about 1 weight percent water, and most preferably less than about 0.1 percent water, based on the total weight of the DME concentrated stream. The methanol concentrated stream, in this embodiment, preferably comprises at least about 80 weight percent methanol, more preferably at least about 90 weight percent methanol, and most preferably at least about 95 weight percent methanol, based on the total weight of the methanol concentrated stream. The methanol concentrated stream preferably comprises less than about 5 weight percent DME, more preferably less than about 1 weight percent DME, and most preferably less than about 0.1 weight percent DME, based on the total weight of the methanol concentrated stream. The methanol concentrated stream preferably comprises less than about 20 weight percent water, more preferably less than about 10 weight percent water, and most preferably less than about 5 weight percent water, based on the total weight of the methanol concentrated stream. In this embodiment, the water concentrated stream preferably comprises at least about 80 weight percent water, more preferably at least about 90 weight percent water, and most preferably at least about 99 weight percent water, based on the total weight of the water concentrated stream. The water concentrated stream in this embodiment also preferably comprises less than about 5 weight percent DME, more preferably less than about 1 weight percent DME, and most preferably less than about 0.1 weight percent DME, based on the total weight of the water concentrated stream. The water concentrated stream in this embodiment preferably comprises less than about 20 weight percent methanol, more preferably less than about 10 weight percent methanol, and most preferably less than about 5 weight percent methanol, based on the total weight of the total weight of the water concentrated stream.

The disposition of the methanol concentrated stream may vary widely. In one preferred embodiment, the methanol concentrated stream, or a portion thereof, is directed to the first reaction zone. Optionally, the methanol concentrated stream is combined with the first feed stream, which in turn is directed to the first reaction zone. Additionally or alternatively, the methanol concentrated stream, or a portion thereof, is sent directly to the first reaction zone without combining the methanol concentrated stream with the first feed stream prior to introduction of the first feed stream into the first reaction zone. By directing the methanol concentrated stream to the first reaction zone, the residual methanol contained therein may be converted to DME for subsequent conversion of the DME to light olefins. For purposes of the present specification and the appended claims, the term “residual methanol” means methanol that has passed through the first reaction zone. Similarly, the term “residual syngas” means syngas which has passed through the first reaction zone. In another embodiment, all or a portion of the methanol concentrated stream is directed to the second reaction zone for conversion of the methanol contained thereof to light olefins and water.

Converting Dimethyl Ether to Light Olefins

Whether formed from methanol or from syngas, the thus formed dimethyl ether preferably is converted to light olefins in a second reaction zone (the second reaction step of the present invention). The second reaction zone preferably is part of an oxygenate to olefins (OTO) reaction system, discussed in more detail hereinafter. In an OTO reaction process, an oxygenated feedstock, in this case a dimethyl ether-containing feedstock, is converted in the presence of a molecular sieve catalyst composition into one or more olefins, preferably and predominantly, ethylene and/or propylene, referred to herein as light olefins. As used herein, “reaction system” means a system comprising a reaction zone, optionally a disengaging zone, optionally a catalyst regenerator, optionally a catalyst cooler and optionally a catalyst stripper.

The second reaction zone preferably receives a second feed stream, which is derived from the first reaction zone. The second feed stream optionally comprises all or a portion of any of the following streams: the first overhead stream, the DME concentrated stream and all or a portion of the methanol concentrated stream, if a methanol concentrated stream was formed.

Preferably, a molecular sieve catalyst is used to convert the dimethyl ether and optionally any methanol contained in the second feed stream to light olefins and water. Thus, the second catalyst used in the step of converting the dimethyl ether to light olefins (the second reaction step) preferably comprises a molecular sieve catalyst composition, which preferably comprises a zeolitic or a non-zeolitic molecular sieve catalyst composition. Ideally, the molecular sieve catalyst composition comprises an alumina or a silica-alumina catalyst composition. Silicoaluminophosphate (SAPO) molecular sieve catalysts are particularly desirable in such conversion processes because they are highly selective in the formation of ethylene and propylene. A non-limiting list of preferable SAPO molecular sieve catalyst compositions includes SAPO-17, SAPO-18, SAPO-34, SAPO-35, SAPO-44, the substituted forms thereof, and mixtures thereof. Preferably, the second catalyst comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof.

Although the present specification is specifically directed to converting dimethyl ether to light olefins in an OTO reaction system, one or more additional components may be included in the second feed stream that is directed to the OTO reaction system. For example, the second feed stream that is directed to the OTO reaction system optionally contains, in addition to dimethyl ether, one or more aliphatic-containing compounds such as alcohols, amines, carbonyl compounds for example aldehydes, ketones and carboxylic acids, ethers, halides, mercaptans, sulfides, and the like, and mixtures thereof. The aliphatic moiety of the aliphatic-containing compounds optionally contains from 1 to about 50 carbon atoms, preferably from 1 to 20 carbon atoms, more preferably from 1 to 10 carbon atoms, and more preferably from 1 to 4 carbon atoms, and most preferably comprises methanol.

Non-limiting examples of aliphatic-containing compounds include: alcohols such as methanol and ethanol, alkyl-mercaptans such as methyl mercaptan and ethyl mercaptan, alkyl-sulfides such as methyl sulfide, alkyl-amines such as methyl amine, alkyl-ethers such as diethyl ether and methyl ethyl ether (in addition to dimethyl ether), alkyl-halides such as methyl chloride and ethyl chloride, alkyl ketones such as dimethyl ketone, alkyl-aldehydes such as formaldehyde and acetaldehyde, and various acids such as acetic acid.

In a preferred embodiment of the process of the invention, the second feed stream contains one or more oxygenates in addition to dimethyl ether or, more specifically, one or more organic compounds containing at least one oxygen atom. In the most preferred embodiment of the process of invention, the oxygenate in the second feed stream (in addition to dimethyl ether) comprises one or more alcohols, preferably aliphatic alcohols where the aliphatic moiety of the alcohol(s) has from 1 to 20 carbon atoms, preferably from 1 to 10 carbon atoms, and most preferably from 1 to 4 carbon atoms. The alcohols useful as feedstock in the process of the invention include lower straight and branched chain aliphatic alcohols and their unsaturated counterparts. Non-limiting examples of oxygenates, in addition to dimethyl ether, include methanol, n-propanol, isopropanol, methyl ethyl ether, diethyl ether, di-isopropyl ether, formaldehyde, dimethyl carbonate, dimethyl ketone, acetic acid, and mixtures thereof. In the most preferred embodiment, the second feed stream comprises dimethyl ether, and one or more of methanol, ethanol, diethyl ether or a combination thereof.

The various possible components in the second feed stream, discussed above, are converted primarily into one or more olefins. The olefins or olefin monomers produced from the feedstock typically have from 2 to 30 carbon atoms, preferably 2 to 8 carbon atoms, more preferably 2 to 6 carbon atoms, still more preferably 2 to 4 carbons atoms, and most preferably ethylene and/or propylene.

Non-limiting examples of olefin monomer(s) include ethylene, propylene, butene-1, pentene-1,4-methyl-pentene-1, hexene-1, octene-1 and decene-1, preferably ethylene, propylene, butene-1, pentene-1,4-methyl-pentene-1, hexene-1, octene-1 and isomers thereof. Other olefin monomers include unsaturated monomers, diolefins having 4 to 18 carbon atoms, conjugated or nonconjugated dienes, polyenes, vinyl monomers and cyclic olefins.

In a preferred embodiment, the second feed stream, which contains dimethyl ether and optionally methanol (as well as one or more other oxygenates identified above), is converted in the presence of a molecular sieve catalyst composition into olefin(s) having 2 to 6 carbons atoms, preferably 2 to 4 carbon atoms. Most preferably, the olefin(s), alone or combination, are converted from a feedstock containing an oxygenate, preferably an alcohol, most preferably methanol, to the preferred olefin(s) ethylene and/or propylene.

The second feed stream, in one embodiment, contains one or more diluents, typically used to reduce the concentration of the feedstock. The diluents are generally non-reactive to the feedstock or molecular sieve catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. In other embodiments, the second feed stream does not contain any diluent.

The diluent may be used either in a liquid or a vapor form, or a combination thereof. The diluent is either added directly to a feedstock entering into the second reaction zone or added directly into a reactor, or added with a molecular sieve catalyst composition. In one embodiment, the amount of diluent in the second feed stream is in the range of from about 1 to about 99 mole percent based on the total number of moles of the oxygenate(s) and diluent, preferably from about 1 to 80 mole percent, more preferably from about 5 to about 50, most preferably from about 5 to about 25. In one embodiment, other hydrocarbons are added to the second feed stream either directly or indirectly, and include olefin(s), paraffin(s), aromatic(s) (see for example U.S. Pat. No. 4,677,242, addition of aromatics) or mixtures thereof, preferably propylene, butylene, pentylene, and other hydrocarbons having 4 or more carbon atoms, or mixtures thereof.

The process for converting the second feed stream, especially a feedstock containing one or more oxygenates, in addition to dimethyl ether, in the presence of a molecular sieve catalyst composition of the invention, is carried out in a reaction process in a reactor, where the process is a fixed bed process, a fluidized bed process (includes a turbulent bed process), preferably a continuous fluidized bed process, and most preferably a continuous high velocity fluidized bed process.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunii and O. Levenspiel, Robert E. Krieger Publishing Company, New York, N.Y. 1977, which are all herein fully incorporated by reference.

The preferred reactor type are riser reactors generally described in Riser Reactor, Fluidization and Fluid-Particle Systems, pages 48 to 59, F. A. Zenz and D. F. Othmer, Reinhold Publishing Corporation, New York, 1960, and U.S. Pat. No. 6,166,282 (fast-fluidized bed reactor), and U.S. patent application Ser. No. 09/564,613 filed May 4, 2000 (multiple riser reactor), which are all herein fully incorporated by reference.

In an embodiment, the amount of liquid feedstock fed separately or jointly with a vapor feedstock, to the second reaction zone is in the range of from 0.1 weight percent to about 85 weight percent, preferably from about 1 weight percent to about 75 weight percent, more preferably from about 5 weight percent to about 65 weight percent based on the total weight of the second feed stream including any diluent contained therein. The liquid and vapor feedstocks are preferably the same composition, or contain varying proportions of the same or different feedstock with the same or different diluent.

The conversion temperature employed in the conversion process, specifically within the second reaction zone, is in the range of from about 392° F. (200° C.) to about 1832° F. (1000° C.), preferably from about 482° F. (250° C.) to about 1472° F. (800° C.), more preferably from about 482° F. (250° C.) to about 1382° F. (750° C.), yet more preferably from about 572° F. (300° C.) to about 1202° F. (650° C.), yet even more preferably from about 662° F. (350° C.) to about 1112° F. (600° C.) most preferably from about 662° F. (350° C.) to about 1022° F. (550° C.).

The conversion pressure employed in the conversion process, specifically within the reactor system, varies over a wide range including autogenous pressure. The conversion pressure is based on the partial pressure of the feedstock exclusive of any diluent therein. Typically the conversion pressure employed in the process is in the range of from about 0.1 kPaa to about 5 MPaa, preferably from about 5 kPaa to about 1 MPaa, and most preferably from about 20 kPaa to about 500 kPaa.

The weight hourly space velocity (WHSV), particularly in a process for converting the dimethyl ether in the second feed stream in the presence of a molecular sieve catalyst composition within the second reaction zone, is defined as the total weight of the second feed stream excluding any diluents to the second reaction zone per hour per weight of molecular sieve in the molecular sieve catalyst composition in the second reaction zone. The WHSV is maintained at a level sufficient to keep the catalyst composition in a fluidized state within a reactor.

Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹, preferably the WHSV for conversion of a feedstock containing DME or both DME and methanol, is in the range of from about 1 hr⁻¹ to about 300 hr⁻¹.

The superficial gas velocity (SGV) of the second feed stream including diluent and reaction products within the second reaction zone is preferably sufficient to fluidize the molecular sieve catalyst composition within a reaction zone in the reactor. The SGV in the process, particularly within the second reaction zone, more particularly within the riser reactor(s), is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec. See for example U.S. patent application Ser. No. 09/708,753 filed Nov. 8, 2000, which is herein incorporated by reference.

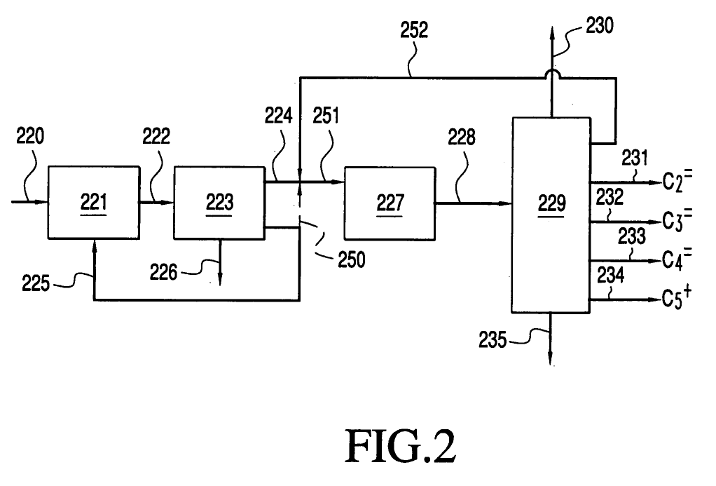

FIG. 2 illustrates one embodiment of the present invention wherein residual oxygenates (methanol and dimethyl ether) from the second effluent stream are recovered and recycled to the second reaction step. As shown, a first feed stream 220 is directed to a first reaction zone 221. In one embodiment, the first feed stream comprises syngas. Additionally or alternatively, the first feed stream 220 comprises methanol. First feed stream 220 may include one or more additional components as identified above. First reaction zone 221, in one embodiment, comprises a reactor, preferably a fixed bed reactor, wherein the syngas and/or methanol contacts a first catalyst under conditions effective to convert the syngas and/or methanol to dimethyl ether and water. The dimethyl ether and water formed in the first reaction zone is yielded therefrom in a first effluent stream 222. If the first effluent stream 222 comprises unreacted residual syngas, then the syngas in the first effluent stream 222 preferably is separated, e.g., by compressing, cooling and separating, and recycled back to the first reaction zone 221 in a syngas recycle stream, not shown. Ideally, the syngas is recycled to extinction and does not pass to the second reaction zone. Additionally, the first effluent stream 222 may comprise unreacted or residual methanol.

As shown, first effluent stream 222 is directed to a first separation zone 223 wherein two or more components contained in the first effluent stream 222 are separated from one another, preferably through distillation or other well known separation techniques. As shown, first separation zone 223 separates the first effluent stream 222 into a DME concentrated stream 224, a methanol concentrated stream 225, and a water concentrated stream 226. Either or both the DME concentrated stream 224 and/or the methanol concentrated stream 225 could be referred to as dewatered streams as water preferably has been at least partially removed from both streams in the formation of water concentrated stream 226. The DME concentrated stream 224 also could be referred to as the “first overhead stream,” and the water concentrated stream 226 could be referred to as the “first bottoms stream” if first separation zone 223 comprises a distillation column.

In another embodiment, not shown, the first separation zone 223 separates the first effluent stream 222 into a DME concentrated stream 224 and a water concentrated stream 226 without forming the separate methanol concentrated stream 225. In this embodiment, a weight majority of the residual methanol contained in the first effluent stream 222 preferably is contained in the DME concentrated stream 224. It is contemplated, however, that a weight majority of the residual methanol contained in the first effluent stream 222 may be separated in the first separation zone 223 into the water concentrated stream 226.

Reverting to FIG. 2, as shown, methanol concentrated stream 225, or a portion thereof, is directed to the first reaction zone 221 as a recycle stream for the conversion to the methanol contained therein to additional dimethyl ether and water. Optionally, the methanol concentrated stream 225, or a portion thereof, is combined with first feed stream 220 prior to the introduction thereof into first reaction zone 221.

DME concentrated stream 224, or a portion thereof, preferably is directed to second reaction zone 227 wherein the DME in the DME concentrated stream 224 contacts a second catalyst under conditions effective to convert the DME to light olefins and water. As shown, DME concentrated stream 224 is combined with an oxygenate recycle stream 252 to form a combined stream 251, which is introduced as the second feed stream into second reaction zone 227. Optionally, the combined stream 251 further comprises all or a portion of methanol concentrated stream 225. In one embodiment, a portion of the methanol concentrated stream 225 is directed to and combined with the DME concentrated stream 224, as shown by broken arrow 250, prior to the introduction thereof into second reaction zone 227 as the combined stream 251. Thus, combined stream 251 optionally comprises all or a portion of DME concentrated stream 224 and optionally one or more of the oxygenate recycle stream 252, and/or methanol concentrated stream 225, or portions thereof.

The light olefins and water formed in the second reaction zone 227 preferably are yielded therefrom in a second effluent stream 228. Second effluent stream 228 may comprise, in addition to the light olefins and water, C4 saturates and olefins and C5+ hydrocarbons. As shown, second effluent stream 228 is directed to a second separation zone 229 for the separation of the various components contained in second effluent stream 228. Second separation zone 229 preferably comprises a series of separation vessels, such as, but not limited to, one or more quench columns, distillation columns, absorption columns, adsorption columns, and one or more compression and knockout drums. As shown, second separation zone 229 separates second effluent stream 228 into a light ends stream 230, an oxygenate recycle stream 252, which preferably comprises unreacted residual DME, methanol, and other oxygenate components. Oxygenate recycle stream 252, or a portion thereof, optionally is directed to and combined with DME concentrated stream 224 to form combined stream 251, as discussed above. Second separation zone 229 also forms an ethylene product stream 231, which preferably comprises polymerization or chemical grade ethylene. Second separation zone 229 also forms a propylene product stream 232, which preferably comprises polymerization or chemical grade propylene. As used herein, “polymer grade” ethylene or propylene comprises at least about 99 weight percent ethylene or propylene, respectively, and “chemical grade” ethylene or propylene comprises at least about 95 weight percent ethylene or propylene, respectively. Ethylene product stream 231 preferably comprises a weight majority of the ethylene present in the second effluent stream 228, and propylene product stream 232 preferably comprises a weight majority of the propylene contained in second effluent stream 228.

Second separation zone 229 also optionally forms a butylene product stream 233 comprising butylene and a C5+ product stream 234 comprising C5+ hydrocarbons. Second separation zone 229 preferably also forms a water stream 235, which preferably comprises a weight majority of the water that was contained in second effluent stream 228.

Preferred Water Separation Processes

In another embodiment, the present invention is to various separation processes in a system for converting syngas and/or methanol to dimethyl ether and water, and converting the dimethyl ether to light olefins and additional water. As water is formed in both the first and second reaction steps of the above-described process, it is advantageous to provide an integrated system for removing water from the reaction system while recycling as much of the residual DME and residual methanol as possible. In one embodiment, the first separation zone, discussed above with reference to FIG. 2, comprises a water removal unit, which receives the first effluent stream from the first reaction zone and one or more water containing streams from the second separation zone. The water removal unit in this embodiment preferably is ideally suited for separating residual oxygenate components such as residual DME and residual methanol from the water received therein.

In one embodiment, the present invention is directed to a process for forming light olefins wherein at least a portion of the water formed in the second reaction step is recycled to a first separation zone, which also receives at least a portion of the first effluent stream. Specifically, in this embodiment, the invention comprises the step of contacting methanol with a first catalyst to form a first effluent stream comprising dimethyl ether and water. A recycle stream is added to the first effluent stream to form a combined stream. Water is removed from the combined stream to form a DME concentrated stream comprising dimethyl ether. The dimethyl ether from the DME concentrated stream contacts a second catalyst to form a second effluent stream comprising the light olefins and additional water. The second effluent stream is separated into a product stream and the recycle stream, which is added to the first effluent stream as described above.

In a similar embodiment, the invention is to a process for forming light olefins that includes a step of contacting syngas and optionally recycled methanol with a first catalyst to form a first effluent stream comprising dimethyl ether, methanol and water. A recycle stream is added to the first effluent stream to form a combined stream. Water is removed from the combined stream to form a DME concentrated stream comprising dimethyl ether and methanol. The dimethyl ether from the DME concentrated stream and optionally the methanol from the DME concentrated stream contact a second catalyst to form a second effluent stream comprising the light olefins and additional water. The second effluent stream is separated into a product stream and the recycle stream, which is added to the first effluent stream as discussed above. In this embodiment, the inventive process optionally further comprises the step of separating a weight majority of the dimethyl ether in the DME concentrated stream from a weight majority of the methanol in the DME concentrated stream prior to the step of contacting the dimethyl ether from the DME concentrated stream with the second catalyst. Additionally, the inventive process optionally further comprises the step of recycling the separated methanol from the methanol concentrated stream as the recycled methanol in the step of contacting the syngas and optionally the recycled methanol with the first catalyst.

In this embodiment, the second effluent stream preferably comprises at least about 22 molar percent light olefins, more preferably at least about 32 molar percent light olefins, and most preferably at least about 36 molar percent light olefins, based on the total moles of light olefins and water in the second effluent stream. The recycle stream, which is added to the first effluent stream to form the combined stream, preferably comprises water. Specifically, the recycle stream preferably comprises at least about 70 weight percent water, more preferably at least about 80 weight percent water, and most preferably at least about 90 weight percent water, based on the total weight of the recycle stream. The recycle stream also preferably comprises residual methanol and/or residual dimethyl ether, which can be recovered in the first separation zone. Optionally, the recycle stream comprises at least about 2 weight percent methanol, more preferably at least about 5 weight percent methanol and most preferably at least about 10 weight percent methanol, based on the total weight of the recycle stream. Optionally, the recycle stream comprises at least about 0.1 weight percent dimethyl ether, more preferably at least about 0.5 weight percent dimethyl ether, and most preferably at least about 2 weight percent dimethyl ether, based on the total weight of the recycle stream.

The step of separating the second effluent stream preferably comprises quenching the second effluent stream under conditions effective to form a quench overhead stream and a quench bottoms stream. The quench overhead stream comprises a weight majority of the light olefins and the quench bottom stream comprises a weight majority of the water formed in the step of contacting the dimethyl ether from the DME concentrated stream with the second catalyst. The recycle stream, in this embodiment, ideally comprises at least a portion of the quench bottoms stream.

Additionally or alternatively, the step of separating the second effluent stream comprises compressing at least a portion of the second effluent stream to form a compressed stream and cooling at least a portion of the compressed stream under conditions effective to form a knockout overhead stream and a knockout bottoms stream. The knockout overhead stream in this embodiment comprises a weight majority of the light olefins from the compressed stream and the knockout bottoms stream comprises a weight majority of the water from the compressed stream. The knockout bottoms stream also preferably comprises residual methanol and/or dimethyl ether, which can be recovered in the first separation zone. In this embodiment, the recycle stream comprises at least a portion of the knockout bottoms stream. In one embodiment, the first effluent stream, the combined stream and the DME concentrated stream further comprise residual methanol. In this embodiment, the process optionally further comprises a step of contacting the residual methanol in the DME concentrated stream with the second catalyst under conditions effective to convert the residual methanol to light olefins and water. Additionally or alternatively, the process further comprises the step of separating and recycling a weight majority of the residual methanol from the DME concentrated stream to the first step of contacting the methanol with the first catalyst.

In one embodiment, at least a portion of the water removed from the combined stream is directed to a syngas generation unit, for example to serve as a source of steam for steam reforming.

Optionally, the step of adding the recycle stream to the first effluent stream to form the combined stream and the step of removing water from the combined stream occur in a separation unit. Alternatively, the step of adding the recycle stream to the first effluent stream occurs outside of a separation unit, and the step of removing the water from the combined stream occurs in the separation unit.

In a similar embodiment, the process of the present invention comprises a step of contacting methanol with a first catalyst in a first reaction zone under conditions effective to convert the methanol to dimethyl ether and water. The dimethyl ether, unreacted methanol, the water and a recycle stream are combined to form a combined stream. The combined stream, in this embodiment, is separated into a first overhead stream and a first bottom stream. The first overhead stream comprises a weight majority of the dimethyl ether and a weight majority of the unreacted methanol from the combined stream, and the first bottom stream comprises a weight majority of the water from the combined stream. The dimethyl ether and optionally the unreacted methanol in the first overhead stream contact a second catalyst in a second reaction zone under conditions effective to convert the dimethyl ether and optionally the optional unreacted methanol to the light olefins and water. A portion of the water formed in the step of contacting the dimethyl ether with the second catalyst is removed to form the recycle stream which is combined with the dimethyl ether, the unreacted methanol and the water, as discussed above.

In another embodiment, the process includes a step of contacting syngas and optionally methanol with a first catalyst in a first reaction zone under conditions effective to convert the syngas and optionally the methanol to dimethyl ether, methanol and water. The dimethyl ether, the methanol, the water and a recycle stream are combined to form a combined stream. The combined stream is separated into a first overhead stream and a first bottom stream. The first overhead stream comprises a weight majority of the dimethyl ether and a weight majority of the methanol from the combined stream, and the first bottom stream comprises a weight majority of the water from the combined stream. The dimethyl ether and optionally the methanol in the first overhead stream contact a second catalyst in a second reaction zone under conditions effective to convert the dimethyl ether and the optionally methanol to the light olefins and water. A portion of the water formed in the step of contacting the dimethyl ether with the second catalyst is removed to form the recycle stream.

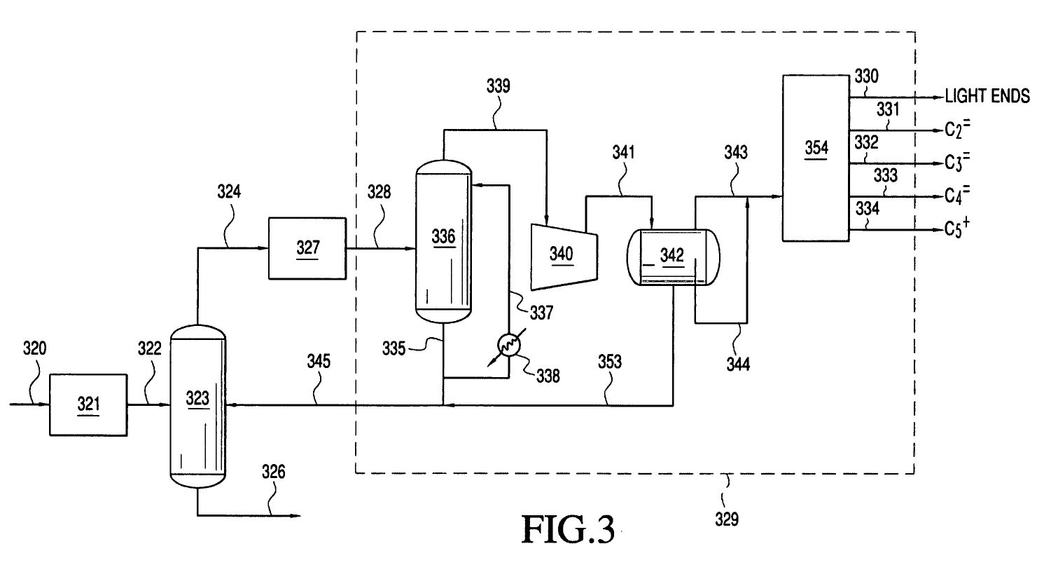

One embodiment of this inventive process is shown in FIG. 3 wherein water and unreacted residual oxygenates (methanol and/or dimethyl ether) from the second effluent stream are recycled to the first separation zone to recover the residual oxygenates. As shown, a first feed stream 320 is directed to a first reaction zone 321. In one embodiment, the first feed stream comprises syngas. Additionally or alternatively, the first feed stream 320 comprises methanol. First feed stream 320 may include one or more additional components as identified above. First reaction zone 321, in one embodiment, comprises a reactor, preferably a fixed bed reactor, wherein the syngas and/or methanol contacts a first catalyst under conditions effective to convert the syngas and/or methanol to dimethyl ether and water. The dimethyl ether and water formed in the first reaction zone is yielded therefrom in a first effluent stream 322. If the first effluent stream 322 comprises unreacted residual syngas, then the syngas in the first effluent stream preferably is separated, e.g., by compressing, cooling and separating, and recycled back to the first reaction zone 321 in a syngas recycle stream, not shown. Ideally, the syngas is recycled to extinction and does not pass to the second reaction zone 327. Additionally, the first effluent stream 322 may comprise unreacted residual methanol.

As shown, first effluent stream 322 is directed to a first separation zone 323 wherein two or more components contained in the first effluent stream 322 are separated from one another, preferably through distillation or other well known separation techniques. As shown, first separation zone 323 separates the first effluent stream 322 into a DME concentrated stream 324, and a water concentrated stream 326. In this embodiment, a weight majority of the residual methanol contained in the first effluent stream 322 preferably is contained in the DME concentrated stream 324. It is contemplated, however, that a weight majority of the residual methanol contained in the first effluent stream 322 may be separated in the first separation zone 323 into the water concentrated stream 326. Optionally, however, the first separation zone 323 also forms a separate methanol concentrated stream, not shown but discussed above with reference to FIG. 2, which comprises a weight majority of the methanol, if any, that was contained in the first effluent stream. The optional methanol concentrated stream or a portion thereof optionally is directed to the first reaction zone as a recycle stream for the conversion to the methanol contained therein to additional dimethyl ether and water. Optionally, the methanol concentrated stream, or a portion thereof, is combined with first feed stream 320 prior to the introduction thereof into first reaction zone 321, as discussed above with reference to the methanol concentrated stream 225 in FIG. 2. Water concentrated stream 326 preferably is directed to a water treatment facility.

DME concentrated stream 324, or a portion thereof, preferably is directed to second reaction zone 327 wherein the DME (and optionally any methanol contained in the DME concentrated stream) in the DME concentrated stream 324 contacts a second catalyst under conditions effective to convert the DME to light olefins and water. Optionally, DME concentrated stream 324 is combined with an oxygenate recycle stream, not shown, to form a combined stream, not shown, as discussed above in FIG. 2. Optionally, the combined stream further comprises all or a portion of optional methanol concentrated stream.

The light olefins and water formed in second reaction zone 327 are yielded therefrom in second effluent stream 328. Second effluent stream 328 may comprise components in addition to the light olefins and water, such as but not limited to unreacted oxygenates (particularly, methanol and/or dimethyl ether) C4 olefins, C5+ hydrocarbons, and light ends. As shown, second effluent stream 328 is directed to a quench unit 336 in second separation zone 329. In quench unit 336, the second effluent stream 328 preferably contacts a quenching medium under conditions effective to condense out the readily condensable components contained in second effluent stream 328. As shown, the second effluent stream 328 is separated in quench unit 336 into a quench overhead stream 339 and a quench bottoms steam 335. Quench overhead stream 339 preferably comprises a weight majority of the ethylene, propylene, butylene, and C5+ components, individually or collectively, that were contained in second effluent stream 328. Quench bottoms stream 335 preferably comprises a weight majority of the water that was contained in second effluent stream 328, based on the total weight of the water in second effluent stream 328. Quench bottoms stream 335 also preferably comprises a weight majority of the residual methanol and some residual dimethyl ether, individually or collectively, that was contained in second effluent stream 328. A portion of quench bottoms stream 335 preferably is recycled to the top of the quench unit 336 as quench medium 337. Quench medium 337 preferably is cooled in heat exchanger 338 prior to being reintroduced into quench unit 336.

Quench bottoms stream 335 may comprise unreacted residual methanol and/or unreacted residual dimethyl ether. In order to recover and reuse this residual methanol and/or residual DME, it is desirable according to the present invention to direct at least a portion of quench bottoms stream 335 to first separation zone 323 for recovery of the residual methanol and/or residual DME contained therein. As shown, the portion of the quench bottom stream 335 that is not recycled to the top of quench unit 336 via quench medium 337 is directed to first separation zone 323 through water recycle stream 345. In another embodiment, water recycle stream 345 is directed to and combined with first effluent stream 322 prior to the introduction thereof into first separation zone 323.

Quench overhead stream 339 preferably is directed to one or more compression units wherein the quench overhead stream 339 is compressed to form compressed stream 341. Compressed stream 341 is directed to one or more knockout drums 342 wherein readily condensable components are separated from non-readily condensable components contained in compressed steam 341. FIG. 3 illustrates a single compression stage comprising compression unit 340 and knockout drum 342. It is contemplated, however, that quench overhead stream 339 may be compressed in a plurality of compression stages, each stage preferably comprising a compression unit and a knockout drum.

Referring to FIG. 3, as shown, compressed stream 341 is separated in knockout drum 342 into knockout overhead stream 343 and knockout bottoms stream 353 and a knockout hydrocarbon stream 344. Knockout bottoms stream 353 preferably comprises a weight majority of water and a minor amount of recovered oxygenates such as residual methanol and/or residual DME. Ideally, the knockout bottoms stream comprises a weight majority of the residual methanol and/or residual dimethyl ether, individually or collectively, that was contained in compressed stream 341. A hydrocarbon layer that accumulates on top of the aqueous layer in drum 342 is removed from knockout drum 342 in knockout hydrocarbon stream 344 and is directed ultimately to separation train 354. All or a portion of knockout bottoms stream 353 preferably is directed to first separation zone 323. As shown, knockout bottoms stream 353 is combined with quench bottoms stream 335 to form water recycle stream 345, which is directed to and introduced into first separation zone 323. In another embodiment, not shown, knockout bottoms stream 353 is introduced into first separation zone 323 in a separate line from quench bottoms stream 335. In another embodiment, not shown, knockout bottoms stream 353 is combined with first effluent stream 322 prior to the introduction thereof into first separation zone 323. In yet another embodiment, not shown, quench bottoms stream 335, or a portion thereof, knockout bottoms stream 353 and first effluent stream 322 are combined to form a single stream which is directed to first separation zone 323 for the removal of water therefrom.

Knockout overhead stream 343 preferably is directed to separation train 354 for the separation of two or more of the components contained therein. Separation train 354 preferably comprises a plurality of separation units such as distillation columns, adsorption columns and/or absorption columns. As shown, separation train 354 separates knockout overhead stream 343 into a light ends stream 330, an ethylene product stream 331, a propylene product stream 332, a butylene product stream 333, and a C5+ hydrocarbon stream 334.

This embodiment of the present invention is particularly advantageous in that it minimizes equipment count and reduces start up and operating costs for the process of converting methanol and/or syngas to light olefins via a dimethyl ether intermediate. Specifically, the embodiment of the present invention shown in FIG. 3 provides for removing the water from the first effluent stream 322, the quench bottoms stream 335 and the knockout bottoms stream 353 in a single separation unit (the first separation zone 323). In addition to minimizing equipment count, the present invention provides an ideal processing scheme for recovering unreacted residual methanol and/or unreacted residual DME from one or both of the quench bottoms stream 335 and the knockout bottoms stream 353. As a result, overall conversion of methanol and/or DME to light olefins can be improved over conventional reaction systems.

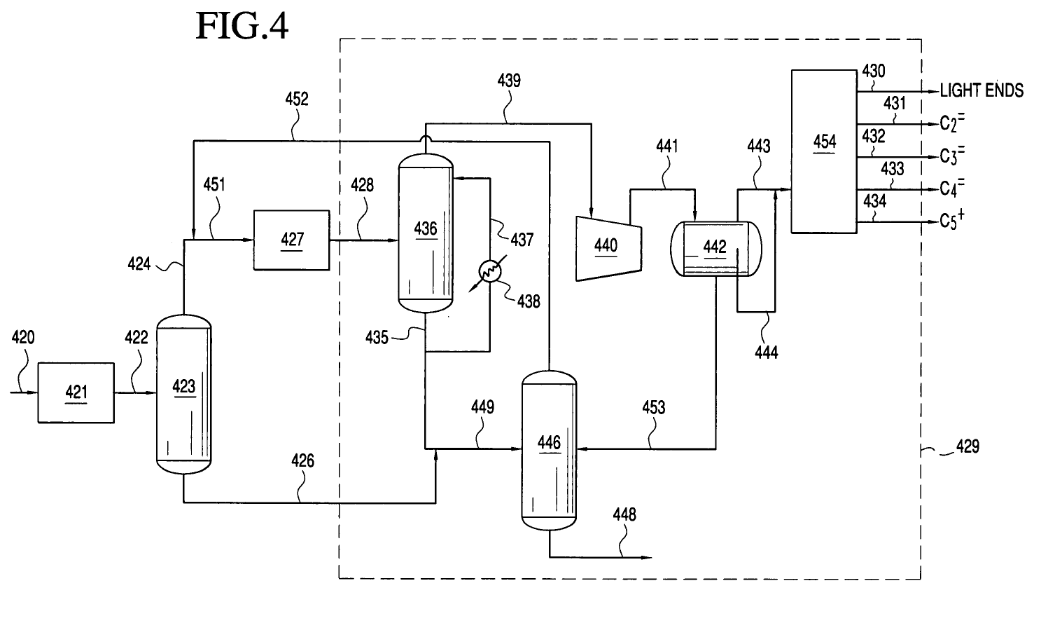

FIG. 4 illustrates another embodiment of the present invention wherein a separate oxygenate recovery unit 446 is provided in the second separation zone 429 to recover oxygenates from a quench bottoms stream 435, a knockout drum bottoms stream 453, and/or a water concentrated stream 426. As shown, a first feed stream 420 is directed to a first reaction zone 421. In one embodiment, the first feed stream comprises syngas. Additionally or alternatively, the first feed stream 420 comprises methanol. First feed stream 420 may include one or more additional components as identified above. First reaction zone 421, in one embodiment, comprises a reactor, preferably a fixed bed reactor, wherein the syngas and/or methanol contacts a first catalyst under conditions effective to convert the syngas and/or methanol to dimethyl ether and water. The dimethyl ether and water formed in the first reaction zone is yielded therefrom in a first effluent stream 422. If the first effluent stream 422 comprises unreacted residual syngas, then the syngas in the first effluent stream 422 preferably is separated, e.g., by compressing, cooling and separating, and recycled back to the first reaction zone 421 in a syngas recycle stream, not shown. Ideally, the syngas is recycled to extinction and does not pass to the second reaction zone 427. Additionally, the first effluent stream 422 may comprise unreacted residual residual methanol.

As shown, first effluent stream 422 is directed to a first separation zone 423 wherein two or more components contained in the first effluent stream 422 are separated from one another, preferably through distillation or other well known separation techniques. As shown, first separation zone 423 separates the first effluent stream 422 into a DME concentrated stream 424, and a water concentrated stream 426. In this embodiment, a weight majority of the residual methanol contained in the first effluent stream 422 preferably is contained in the DME concentrated stream 424. It is contemplated, however, that a weight majority of the residual methanol contained in the first effluent stream 422 may be separated in the first separation zone 423 into the water concentrated stream 426. Optionally, however, the first separation zone 423 also forms a separate methanol concentrated stream, not shown but discussed above with reference to FIG. 2, which comprises a weight majority of the methanol, if any, that was contained in the first effluent stream. The optional methanol concentrated stream or a portion thereof optionally is directed to the first reaction zone as a recycle stream for the conversion to the methanol contained therein to additional dimethyl ether and water. Optionally, the methanol concentrated stream, or a portion thereof, is combined with first feed stream 420 prior to the introduction thereof into first reaction zone 421, as discussed above with reference to FIG. 2.

DME concentrated stream 424, or a portion thereof, preferably is combined with an oxygenate recycle stream 452 to form combined stream 451, which is directed to second reaction zone 427. Optionally, the combined stream 451 further comprises all or a portion of optional methanol concentrated stream as discussed above in reference to FIG. 2. In second reaction zone 427, the DME (and optionally any methanol contained in combined stream 451) in the combined stream 451 contacts a second catalyst under conditions effective to convert the DME to light olefins and water.

The light olefins and water formed in second reaction zone 427 are yielded therefrom in second effluent stream 428. Second effluent stream 428 may comprise components in addition to the light olefins and water, such as but not limited to unreacted oxygenates (particularly, methanol and/or dimethyl ether) C4 olefins, C5+ hydrocarbons, and light ends. As shown, second effluent stream 428 is directed to a quench unit 436 in second separation zone 429. In quench unit 436, the second effluent stream 428 preferably contacts a quenching medium under conditions effective to condense out the readily condensable components contained in second effluent stream 428. As shown, the second effluent stream 428 is separated in quench unit 436 into a quench overhead stream 439 and a quench bottoms steam 435. Quench overhead stream 439 preferably comprises a weight majority of the ethylene, propylene, butylene, and C5+ components, individually or collectively, that were contained in second effluent stream 428. Quench bottoms stream 435 preferably comprises a weight majority of the water that was contained in second effluent stream 428, based on the total weight of the water in second effluent stream 428. Quench bottoms stream 435 also preferably comprises a weight majority of the residual methanol and/or residual dimethyl ether, individually or collectively, that was contained in second effluent stream 428. A portion of quench bottoms stream 435 preferably is recycled to the top of the quench unit 436 as quench medium 437. Quench medium 437 preferably is cooled in heat exchanger 438 prior to being reintroduced into quench unit 436.

Quench bottoms stream 435 may comprise unreacted residual methanol and/or unreacted residual dimethyl ether. In order to recover and reuse this residual methanol and/or residual DME, it is desirable according to this embodiment to direct at least a portion of quench bottoms stream 435 to an oxygenate recovery unit 446. Optionally, all or a portion of water concentrated stream 426 also is directed to oxygenate recovery unit 446. This embodiment is particularly preferred if the water concentrated stream 426 comprises dimethyl ether and/or methanol in addition to water. As shown, water concentrated stream 426 is added to and combined with the portion of quench bottom stream 435 that is directed to oxygenate recovery unit 446 to form combined stream 449. Alternatively, water concentrated stream 426 is added directly to oxygenate recovery unit 446 without being introduced into or combined with quench bottom stream 435.

Oxygenate recovery unit 446 acts to recover unreacted residual DME and/or unreacted residual methanol from one or more of the quench bottoms stream 435, the water concentrated stream 426 and/or the knockout bottoms stream 453. As shown, oxygenate recovery unit 446 separates one or more of these streams into an oxygenate recycle stream 452 and a waste water stream 448. The waste water stream 448 preferably is directed to a water treatment facility. Oxygenate recycle stream 452 preferably comprises a weight majority of the residual methanol and/or residual dimethyl ether, individually or collectively, that were contained in the stream(s) that were directed to the oxygenate recovery unit 446, whether the quench bottoms stream 435, the water concentrated stream 426, the knock out bottoms stream 453, or the various combinations thereof. As discussed above, all or portion of oxygenate recycle stream 452 preferably is directed to and optionally combined with quench overhead stream 424 to form combined stream 451. Additionally or alternatively, all or a portion of oxygenate recycle stream 452 is added directly to the second reaction zone 427. In another embodiment, not shown, all or a portion of oxygenate recycle stream 452 is directed to first reaction zone 421 for further conversion to DME. In another embodiment, not shown, all or a portion of oxygenate recycle stream 452 is directed to and combined with first feed stream 420.

Quench overhead stream 439 preferably is directed to one or more compression units wherein the quench overhead stream 439 is compressed to form compressed steam 441. Compressed stream 441 is directed to one or more knockout drums 442 wherein readily condensable components are separated from non-readily condensable components contained in compressed steam 441. FIG. 4 illustrates a single compression stage comprising compression unit 440 and knockout drum 442. It is contemplated, however, that quench overhead stream 439 may be compressed in a plurality of compression stages, each stage preferably comprising a compression unit and a knockout drum.

Referring to FIG. 4, as shown, compressed stream 441 is separated in knockout drum 442 into knockout overhead stream 443, knockout hydrocarbon stream 444, and knockout bottoms stream 453. Knockout bottoms stream 453 preferably comprises a weight majority of water and a minor amount of recovered oxygenates such as residual methanol and/or residual DME. Ideally, the knockout bottoms stream 453 comprises a weight majority of the residual methanol and/or residual dimethyl ether, individually or collectively, that was contained in compressed stream 441. All or a portion of knockout bottoms stream 453 preferably also is directed to oxygenate recovery unit 446. As shown, knockout bottoms stream 453 is added directly to oxygenate recovery unit 446. In another embodiment, not shown, knockout bottoms stream 453 is combined with one or more of quench bottoms stream 435, water concentrated stream 426 and/or combined stream 449, prior to their introduction into oxygenate recovery unit 446. A hydrocarbon layer that accumulates on top of the aqueous layer in knockout drum 442 is removed from the knockout drum 442 in knockout hydrocarbon stream 444 and is directed ultimately to separation train 454.

Knockout overhead stream 443 preferably is directed to separation train 454 for the separation of two or more of the components contained therein. Separation train 454 preferably comprises a plurality of separation units such as distillation columns, adsorption columns and/or absorption columns. As shown, separation train 454 separates knockout overhead stream 443 into a light ends stream 430, an ethylene product stream 431, a propylene product stream 432, a butylene product stream 433, and a C5+ hydrocarbon stream 434.

Debottlenecking Existing MTO Reaction Systems

One embodiment of the present invention is directed to a process for debottlenecking an existing methanol to olefin (MTO) reaction system. By “debottlenecking” it is meant that the net production of light olefins per pound of effluent formed in an OTO reaction system can be advantageously increased. As a result, an OTO reaction system modified according to the present invention can produce more light olefins per pound of effluent thereby providing a commensurate increase in valuable light olefins formed. By “existing methanol to olefins reaction system” it is meant a reaction system that previously received a feedstock whose major oxygenate-containing species by weight was methanol.

In one particular embodiment, the invention is to a process for debottlenecking an existing MTO reaction system. The process includes a step of adding a methanol dehydration reactor to an existing MTO reaction system and converting methanol to dimethyl ether and water in the dehydration reactor. The resulting dimethyl ether contacts a molecular sieve catalyst composition under conditions effective to convert the dimethyl ether to light olefins and water. This step of contacting the dimethyl ether to the molecular sieve catalyst composition preferably occurs in the previously existing MTO reaction system. The light olefins and water formed in the step of contacting the dimethyl ether with the molecular sieve catalyst composition are preferably yielded therefrom in an effluent stream.

This embodiment of the present invention may result in at least a 10 molar percent reduction, preferably at least 20 molar percent reduction, and most preferably at least a 30 molar percent reduction in effluent volumetric flow rate compared to the existing MTO reaction system producing an equal amount of light olefin product.

The effluent stream formed in this embodiment of the present invention preferably has a molar ratio of total effluent stream to light olefins contained therein of less than about 4.5, preferably less than 4.0, and most preferably less than about 3.5.

Having fully described the invention, it will be appreciated by those skilled in the art that the invention may be performed within a wide range of parameters within what is claims, without departing from the spirit and scope of the invention.

We claim:

1. A process for forming light olefins, wherein the process comprises the steps of:
(a) contacting methanol with a first catalyst in a first reaction zone under conditions effective to convert the methanol to dimethyl ether and water; and
(b) contacting the dimethyl ether with a second catalyst in a second reaction zone under conditions effective to convert the dimethyl ether to the light olefins and water.

2. The process of claim 1, wherein the process further comprises the step of:
(a) separating, prior to step (b), a weight majority of the dimethyl ether formed in step (a) from a weight majority of the water formed in step (a).

3. The process of claim 1, wherein the first catalyst comprises a component selected from the group consisting of: an acidic γ-alumina, a modified zeolite, mordenite, a zeolite, ZSM-5, sulfonic acid ion exchange resin and a perfluorinated sulfonic acid ionomer.

4. The process of claim 1, wherein the second catalyst comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof.

5. The process of claim 1, wherein the first reaction zone is in a fixed bed reactor.

6. The process of claim 1, wherein the second reaction zone is in a fluidized reactor.

7. The process of claim 1, wherein the methanol is directed to the first reaction zone in a first feed stream, which further comprises water.

8. A process for forming light olefins, wherein the process comprises the steps of:
(a) contacting syngas with a first catalyst in a first reaction zone under conditions effective to convert the syngas to dimethyl ether, methanol and water; and
(b) contacting the dimethyl ether with a second catalyst in a second reaction zone under conditions effective to convert the dimethyl ether to the light olefins and water.

9. The process of claim 8, wherein the process further comprises the step of:
(a) separating, prior to step (b), a weight majority of the dimethyl ether and the methanol formed in step (a), from a weight majority of the water formed in step (a).

10. The process of claim 8, wherein the process further comprises the step of:
(a) separating, prior to step (b), a weight majority of the dimethyl ether formed in step (a) from a weight majority of the methanol and water formed in step (a).

11. The process of claim 8, wherein the first catalyst comprises a component selected from the group consisting of: an aluminum phosphate (AlPO₄), an acidic alumina, a modified zeolite, mordenite, a zeolite, ZSM-5, sulfonic acid ion exchange resin, a perfluorinated sulfonic acid ionomer, and a copper/zinc oxide combined in a mixture or separate stages.

12. The process of claim 8, wherein the second catalyst comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof.

13. The process of claim 8, wherein the first reaction zone is in a fixed bed reactor.

14. The process of claim 8, wherein the second reaction zone is in a fluidized reactor.

15. A process for forming light olefins, wherein the process comprises the steps of:
(a) contacting methanol with a first catalyst to form a first effluent stream comprising dimethyl ether, methanol, and water;
(b) adding a recycle stream to the first effluent stream to form a combined stream;
(c) removing water from the combined stream to form a DME concentrated stream comprising dimethyl ether and methanol;
(d) contacting the dimethyl ether from the DME concentrated stream with a second catalyst to form a second effluent stream comprising the light olefins and additional water; and
(e) separating the second effluent stream into a product stream and the recycle stream.

16. The process of claim 15, wherein the second effluent stream comprises at least about 22 molar percent light olefins, based on the total moles of light olefins and water in the second effluent stream.

17. The process of claim 16, wherein the second effluent stream comprises at least about 32 molar percent light olefins, based on the total moles of light olefins and water in the second effluent stream.

18. The process of claim 17, wherein the second effluent stream comprises at least about 36 molar percent light olefins, based on the total moles of light olefins and water in the second effluent stream.

19. The process of claim 15, wherein the recycle stream comprises water.

20. The process of claim 15, wherein step (e) comprises quenching the second effluent stream under conditions effective to form an overhead stream and a bottoms stream, wherein the overhead stream comprises a weight majority of the light olefins, and the bottoms stream comprises a weight majority of the water formed in step (d), wherein the recycle stream comprises at least a portion of the bottoms stream.

21. The process of claim 15, wherein step (e) comprises:
(i) compressing at least a portion of the second effluent stream to form a compressed stream;
(ii) cooling at least a portion of the compressed stream under conditions effective to form an overhead stream and a bottoms stream, wherein the overhead stream comprises a weight majority of the light olefins from the compressed stream, and the bottoms stream comprises a weight majority of the water from the compressed stream, wherein the recycle stream comprises at least a portion of the bottoms stream.

22. The process of claim 15, wherein the first effluent stream, the combined stream and the DME concentrated stream further comprise residual methanol, and wherein the process further comprises the step of:
(a) contacting the residual methanol in the DME concentrated stream with the second catalyst under conditions effective to convert the residual methanol to light olefins and water.

23. The process of claim 15, wherein the first effluent stream, the combined stream and the DME concentrated stream further comprise residual methanol, and wherein the process further comprises the step of:
(a) separating and recycling a weight majority of the residual methanol from the DME concentrated stream to step (a).

24. The process of claim 15, wherein at least a portion of the water removed in step (c) is directed to a syngas generation unit.

25. The process of claim 15, wherein the first catalyst comprises a component selected from the group consisting of: an acidic γ-alumina, a modified zeolite, mordenite, a zeolite, ZSM-5, sulfonic acid ion exchange resin and a perfluorinated sulfonic acid ionomer.

26. The process of claim 15, wherein the second catalyst comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof.

27. The process of claim 15, wherein step (a) occurs in a fixed bed reactor.

28. The process of claim 15, wherein step (d) occurs in a fluidized reactor.

29. The process of claim 15, wherein steps (b) and (c) occur in a separation unit.

30. The process of claim 15, wherein step (b) occurs outside of a separation unit, and wherein step (c) occurs in the separation unit.

31. The process of claim 15, wherein the DME concentrated stream comprises at least about 50 weight percent dimethyl ether, based on the total weight of the DME concentrated stream.

32. The process of claim 31, wherein the DME concentrated stream comprises at least about 60 weight percent dimethyl ether, based on the total weight of the DME concentrated stream.

33. The process of claim 32, wherein the DME concentrated stream comprises at least about 70 weight percent dimethyl ether, based on the total weight of the DME concentrated stream.

34. A process for forming light olefins, wherein the process comprises the steps of:
(a) contacting syngas and optionally recycled methanol with a first catalyst to form a first effluent stream comprising dimethyl ether, methanol and water;
(b) adding a recycle stream to the first effluent stream to form a combined stream;
(c) removing water from the combined stream to form a DME concentrated stream comprising dimethyl ether and methanol;
(d) contacting the dimethyl ether from the DME concentrated stream and optionally the methanol from the DME concentrated stream with a second catalyst to form a second effluent stream comprising the light olefins and additional water; and
(e) separating the second effluent stream into a product stream and the recycle stream, which is added in step (b).

35. The process of claim 34, wherein the second effluent stream comprises at least about 22 molar percent light olefins, based on the total moles of light olefins and water in the second effluent stream.

36. The process of claim 35, wherein the second effluent stream comprises at least about 32 molar percent light olefins, based on the total moles of light olefins and water in the second effluent stream.

37. The process of claim 36, wherein the second effluent stream comprises at least about 36 molar percent light olefins, based on the total moles of light olefins and water in the second effluent stream.

38. The process of claim 34, wherein the process further comprises the step of:
(a) separating a weight majority of the dimethyl ether in the DME concentrated stream from a weight majority of the methanol in the DME concentrated stream prior to step (d).

39. The process of claim 38, wherein the process further comprises the step of:
(a) recycling the separated methanol from the DME concentrated stream to step (a) as the recycled methanol.

40. The process of claim 34, wherein the recycle stream comprises water.

41. The process of claim 34, wherein step (e) comprises quenching the second effluent stream under conditions effective to form an overhead stream and a bottoms stream, wherein the overhead stream comprises a weight majority of the light olefins formed in step (d), and the bottoms stream comprises a weight majority of the water formed in step (d), wherein the recycle stream comprises at least a portion of the bottoms stream.

42. The process of claim 34, wherein step (e) comprises:
(i) compressing at least a portion of the second effluent stream to form a compressed stream;
(ii) cooling at least a portion of the compressed stream under conditions effective to form an overhead stream and a bottoms stream, wherein the overhead stream comprises a weight majority of the light olefins from the compressed stream, and the bottoms stream comprises a weight majority of the water from the compressed stream, wherein the recycle stream comprises at least a portion of the bottoms stream.

43. The process of claim 34, wherein at least a portion of the water removed in step (c) is directed to a syngas generation unit.

44. The process of claim 34, wherein the first catalyst comprises a component selected from the group consisting of: an aluminum phosphate (AlPO₄), an acidic γ-alumina, a modified zeolite, mordenite, a zeolite, ZSM-5, sulfonic acid ion exchange resin, a perfluorinated sulfonic acid ionomer, and a copper/zinc oxide combined in a mixture or separate stages.

45. The process of claim 34, wherein the second catalyst comprises a molecular sieve selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ZSM-5, metal containing forms thereof, intergrown forms thereof, AEI/CHA intergrowths, and mixtures thereof.

46. The process of claim 34, wherein step (a) occurs in a fixed bed reactor.

47. The process of claim 34, wherein step (d) occurs in a fluidized reactor.

48. The process of claim 34, wherein steps (b) and (c) occur in a separation unit.

49. The process of claim 34, wherein step (b) occurs outside of a separation unit, and wherein step (c) occurs in the separation unit.

50. The process of claim 34, wherein the first effluent stream comprises at least about 40 weight percent dimethyl ether, based on the total weight of the first effluent stream.

51. The process of claim 50, wherein the first effluent stream comprises at least about 50 weight percent dimethyl ether, based on the total weight of the first effluent stream.

52. The process of claim 51, wherein the first effluent stream comprises at least about 60 weight percent dimethyl ether, based on the total weight of the first effluent stream.

53. The process of claim 34, wherein the DME concentrated stream comprises at least about 50 weight percent dimethyl ether, based on the total weight of the DME concentrated stream.

54. The process of claim 53, wherein the DME concentrated stream comprises at least about 75 weight percent dimethyl ether, based on the total weight of the DME concentrated stream.

55. The process of claim 54, wherein the DME concentrated stream comprises at least about 85 weight percent dimethyl ether, based on the total weight of the DME concentrated stream.

56. A process for forming light olefins, wherein the process comprises the steps of:
(a) contacting methanol with a first catalyst in a first reaction zone under conditions effective to convert the methanol to dimethyl ether and water;
(b) combining the dimethyl ether, unreacted methanol, the water and a recycle stream to form a combined stream;
(c) separating the combined stream into a first overhead stream and a first bottoms stream, wherein the first overhead stream comprises a weight majority of the dimethyl ether and a weight majority of the unreacted methanol from the combined stream, and the first bottoms stream comprises a weight majority of the water from the combined stream;
(d) contacting the dimethyl ether and optionally the unreacted methanol in the first overhead stream with a second catalyst in a second reaction zone under conditions effective to convert the dimethyl ether and optionally the optional unreacted methanol to the light olefins and water; and
(e) removing a portion of the water formed in step (d) to form the recycle stream.

57. A process for forming light olefins, wherein the process comprises the steps of:
(a) contacting syngas and optionally methanol with a first catalyst in a first reaction zone under conditions effective to convert the syngas and optionally the methanol to dimethyl ether, methanol and water;
(b) combining the dimethyl ether, the methanol, the water and a recycle stream to form a combined stream;
(c) separating the combined stream into a first overhead stream and a first bottoms stream, wherein the first overhead stream comprises a weight majority of the dimethyl ether and a weight majority of the methanol from the combined stream, and the first bottoms stream comprises a weight majority of the water from the combined stream;
(d) contacting the dimethyl ether and optionally the methanol in the first overhead stream with a second catalyst in a second reaction zone under conditions effective to convert the dimethyl ether and the optional methanol to the light olefins and water; and
(e) removing a portion of the water formed in step (d) to form the recycle stream.

58. A process for debottlenecking an existing methanol to olefins reaction system, wherein the process comprises the steps of:
(a) adding a methanol dehydration reactor to the existing methanol to olefins reaction system;
(b) converting methanol to dimethyl ether and water in the dehydration reactor;
(c) contacting the dimethyl ether with a molecular sieve catalyst composition under conditions effective to convert the dimethyl ether to light olefins and water; and
(d) yielding the light olefins and water from the reaction system in an effluent stream.

59. The process of claim 58, wherein the process results in at least a 10 molar percent reduction in effluent volumetric flow rate compared to the existing methanol to olefins reaction system.

60. The process of claim 59, wherein the process results in at least a 20 molar percent reduction in effluent volumetric flow rate compared to the existing methanol to olefins reaction system.

61. The process of claim 60, wherein the process results in at least a 30 molar percent reduction in effluent volumetric flow rate compared to the existing methanol to olefins reaction system.

62. The process of claim 58, wherein the effluent stream has a molar ratio of total effluent stream to light olefins contained therein of less than about 4.5.

63. The process of claim 62, wherein the effluent stream has a molar ratio of total effluent stream to light olefins contained therein of less than about 4.0.

64. The process of claim 63, wherein the effluent stream has a molar ratio of total effluent stream to light olefins contained therein of less than about 3.5.
